(19) United States
(12) Patent Application Publication
(10) Pub. No.: US 2010/0240870 A1
(43) Pub. Date: Sep. 23, 2010

(54) Composite organic-inorganic nanoclusters

(76) Inventors: Xing Su, Cupertino, CA (US); Jingwu Zhang, Santa Clara, CA (US); Lei Sun, Santa Clara, CA (US); Andrew A. Berlin, San Jose, CA (US)

Correspondence Address: INTEL CORPORATION;c/o CPA Global, P.O. BOX 52050, MINNEAPOLIS, MN, 55402, US

(21) Appl. No.: 12/658,414

(22) Filed: Feb. 10, 2010

Related U.S. Application Data
(62) Division of application No. 11/021,682, filed on Dec. 23, 2004, now abandoned.
Application No. 11/021,682 is a continuation-in-part of application No. 10/830,422, filed on Apr. 21, 2004, now abandoned.
Application No. 10/830,422 is a continuation-in-part of application No. 10/748,336, filed on Dec. 29, 2003, now abandoned.

(51) Int. Cl. C07K 14/765; C07F 1/10; C07F 15/00; C07F 5/06; C07F 1/12; C07F 1/08; C07K 16/00; C07K 14/00; C07H 21/00; C07H 23/00

(52) U.S. Cl. 530/363; 556/110; 556/136; 556/170; 530/391.1; 530/350; 536/23.1; 536/121; 977/773

(57) ABSTRACT

Composite organic-inorganic nanoclusters (COINs) are provided that produce surface-enhanced Raman signals (SERS) when excited by a laser. The nanoclusters include metal particles and a Raman-active organic compound. The metal required for achieving a suitable SERS signal is inherent in the nanocluster and a wide variety of Raman-active organic compounds and combinations thereof can be incorporated into the nanocluster. In addition, polymeric microspheres containing the nanoclusters and methods of making them are also provided. The nanoclusters and microspheres can be used, for example, in assays for multiplex detection of biological molecules.

### CROSS REFERENCE TO RELATED APPLICATIONS

The present application is a divisional of U.S. patent application Ser. No. 11/021,682, filed on Dec. 23, 2004, now pending, which is a continuation-in-part of U.S. patent application Ser. No.10/830,422, filed on Apr. 21, 2004, now abandoned which is a continuation-in-part of U.S. patent application Ser. No.10/748,336, filed on Dec. 29, 2003, now abandoned, the disclosures of which are considered part of and are incorporated by reference in the disclosure of this application.

### BACKGROUND OF THE INVENTION

1. Field of the Invention

The invention relates generally to nanoclusters that include metal particles and organic compounds and to the use of such nanoclusters in analyte detection by surface-enhanced Raman spectroscopy.

2. Background Information

Multiplex reactions are parallel processes that exist naturally in the physical and biological worlds. When this principle is applied to increase efficiencies of biochemical or clinical analyses, the principal challenge is to develop a probe identification system that has distinguishable components for each individual analyte in a large group of analytes. High density DNA chips and microarrays are probe identification systems in which physical positions on a solid surface are used to identify nucleic acid or protein probes.

In addition, the ability to detect and identify trace quantities of analytes has become increasingly important in virtually every scientific discipline, ranging from part per billion analyses of pollutants in sub-surface water to analysis of cancer treatment drugs in blood serum. Raman spectroscopy is one analytical technique that provides rich optical-spectral information, and surface-enhanced Raman spectroscopy (SERS) has proven to be one of the most sensitive methods for performing quantitative and qualitative analyses. A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity.

Among many analytical techniques that can be used for chemical structure analysis, Raman spectroscopy is attractive for its capability to provide rich structure information from a small optically-focused area or detection cavity. Compared to a fluorescent spectrum that normally has a single peak with half peak width of tens of nanometers to hundreds of nanometers, a Raman spectrum has multiple bonding-structure-related peaks with half peak width of as small as a few nanometers. Furthermore, surface enhanced Raman scattering (SERS) techniques make it possible to obtain a 10⁶ to 10¹⁴ fold Raman signal enhancement. Such huge enhancement factors are attributed primarily to enhanced electromagnetic fields on curved surfaces of coinage metals. Although the electromagnetic enhancement (EME) has been shown to be related to the roughness of metal surfaces or particle size when individual metal colloids are used, SERS is most effectively detected from aggregated colloids. It is known that chemical enhancement can also be obtained by placing molecules in a close proximity to the surface in certain orientations.

Analyses for numerous chemicals and biochemicals by SERS have been demonstrated using: (1) activated electrodes in electrolytic cells; (2) activated silver and gold colloid reagents; and (3) activated silver and gold substrates. None of the foregoing techniques is capable of providing quantitative measurements, however. Consequently SERS has not gained widespread use. In addition, many biomolecules such as proteins and nucleic acids do not have unique Raman signatures because these types of molecules are generally composed of a limited number of common monomers.

SERS effect is attributed mainly to electromagnetic field enhancement and chemical enhancement. It has been reported that silver particle sizes within the range of 50-100 nm are most effective for SERS. Theoretical and experimental studies also reveal that metal particle junctions are the sites for efficient SERS.

### BRIEF DESCRIPTION OF THE FIGURES

FIG. 1 illustrates a method whereby SERS can be used as an amplification method to detect target molecules “A” and “B” according to their Raman signatures and compares this method to the use of COINs containing molecules “A” and “B” to detect molecules “C” and “D.”

FIGS. 2A and B are graphs showing absorption spectra and Raman activity of COINs (composite organic inorganic nanoclusters) made from silver colloids (50 mL) with average particle diameter of 12 nm synthesized with 8-aza-adenine (AA) after 1:30 dilution with sodium citrate. FIG. 2A shows absorption spectra of sample aliquots (1 mL) retrieved from a 95° C. solution at indicated times, showing peak shifts and increased absorption at higher wavelengths (greater than 450 nm). Small arrows indicate positions where absorption changes were further analyzed. Insert shows darkening of samples with time of heat exposure. FIG. 2B is a graph showing absorbance and Raman activity as a function of reaction (heating) time. The Y axis values were in arbitrary units after being normalized to respective maximums; the absorbance ratios of 420 nm/395 nm were used to monitor the shift of the main absorption peak (395 nm 420 nm). Raman scattering signals were measured directly from the same diluted samples without using a salt to induce colloid aggregation. The decrease in absorbance at 700 nm after 65 min. was caused by the formation of large aggregates that settled quickly in solution.

FIGS. 3A-D provide a comparison of Raman signals of SERS and COIN. For each SERS test, 100 μL silver colloid including 4 μM 8-aza-adenine (AA) was mixed with 100 μL of a test reagent chosen from the following: water (control), N-benzoyl adenine (BA, 10 μM); BSA (1%); Tween-203 (Twn, 1%); ethanol (Eth, 100%). A resulting 200 μl mixture was then mixed with either 100 μL it water (−Li,) or 100 μL of 0.34 M LiCl (+Li) before Raman signal was measured. Raman signal intensities were in arbitrary unit and normalized to respective maximums. The same procedure was used for COIN (made with 20 μM 8-aza-adenine) tests, except that additional 8-aza-adenine was not used. FIG. 3A shows Raman spectra of 8-aza-adenine with water as the test reagent, showing salt was required and multiple major peaks were detected; arrows indicate peaks that were stronger than those in COINs. FIG. 3B shows Raman spectra from COINs using water as the test reagent, arrows indicate the reduced peaks compared with those from SERS. FIG. 3C shows bar graphs of SERS signal intensities at 1340 cm⁻¹ under different testing conditions. FIG. 3D shows bar graphs of COIN signal intensities at 1340 cm⁻¹ under different testing conditions.

FIGS. 4A and B show COIN signatures in multiplex detection. COINs were made with individual or mixtures of Raman labels at concentrations from 2.5 μM to 20 μM, depending on signatures desired: 8-aza-adenine (AA), 9-aminoacridine (AN), methylene blue (MB). Representative peaks are indicated by arrows; peak intensity values have been normalized to respective maximums; the Y axis values are in arbitrary units; spectra are offset by 1 unit from each other. FIG. 4A shows signatures of COINs made with the three Raman labels, respectively, showing that each label produced a unique signature. FIG. 4B shows signatures of COINs made from mixtures of the 3 Raman labels at concentrations that produced signatures as indicated: HLL means high peak intensity for AA (H) and low peak intensity for both AN (L) and MB (L); LHL means low peak intensity for AA (L), high peak intensity for AN (H) and Low for MB (L); LLH means low for both AA (L) and AN (L) and high for MB (H). Note that peak heights could be adjusted by varying label concentrations, but they might not necessarily be proportional to label concentrations used due to different adsorption affinity of the Raman labels on metal surfaces.

FIGS. 5A-C illustrate use of COINs as tags for multiplex analyte detection. FIG. 5A is an example detection scheme showing analyte binding by antibody-conjugated. COINs. FIG. 5B shows a set of 50 spectra collected from an immuno sandwich assay for IL-2 using 8-aza-adenine COINs as the tag (main peak position at 1340 cm⁻¹). Background signals were subtracted; spectra were offset in both X and Y axes to show individual spectra. FIG. 5C is a bar graph of analyte signals; the analytes were IL-2 and IL-8 (both having molecular weights of about 20 kDa); experiments were carried out with samples containing 1 or 2 of the analytes at different ratios (5:0, 4:1, 1:1, 1:4, and 0:5). IL-2 detection antibody was conjugated to COIN prepared with 8-aza-adenine (AA) and IL-8 detection antibody was conjugated to COIN made with N-benzoyl adenine (BA). They were used in a 1:1 ratio; data were collected from a total of 400 data points for each sample; spectra showing positive signals at the expected Raman shift positions were counted as measured signal points (wide bars), and expressed as percentages of the total positive signals for both analytes in corresponding samples. The narrow bars indicate expected values (a total of 100% for the 2 labels).

FIGS. 6A and B are graphs showing organic label induced aggregation of metal particles (gold of 15 nm, Abs_{520 nm}=0.37; silver of 60 nm, Abs_{420 nm}=0.3 in 1 mM Na₃Citrate). Each organic compound (see key to abbreviations in Table 1) was mixed with a sample of a metal colloid solution at indicated concentrations for 10 min. before spectral measurement. For each sample, the absorbance of the main peak was used as the Peak 1 value and the increased absorbance at a higher wavelength (600 nm-700 nm) was used as the Peak 2 value; the ratios of Peak 2/Peak 1 were plotted against concentrations of the organic compound; a high value of the ratio indicates a high degree of metal particle aggregation.

FIGS. 7A and B show, respectively, the zeta potential measurements of silver particles of initial z-average size of 47 nm (0.10 mM) with a suspending medium of 1.00 mM sodium citrate and evolution of aggregate size (z-average) in the presence of 20 μM 8-aza-adenine.

FIGS. 8A-D show comparisons of SERS spectra with COIN spectra. Examples of organic compounds as indicated were used for COIN synthesis; the chemical structures of 8 Raman labels are shown. Raman spectra of COINs (C) were overlaid with spectra obtained from SERS (S), showing COIN spectra can have different major peaks compared with respective SERS. In some cases, some peaks were broadened in COINs; spectra were normalized to respective maximums (in arbitrary unit) to show relative peak intensities; note that the main features of spectra were not analyte concentration-dependent.

FIGS. 9A-H show comparisons of Raman signals of SERS and COIN. For SERS tests, silver colloids containing 8-aza-adenine were mixed with a test reagent and then mixed either with water (−Li) or LiCl (+Li) before the Raman scattering signal was measured. The same procedure was used for COIN containing 8-aza-adenine. BA=N-benzoyl adenine; BSA=bovine serum albumin; Twn=Tween-20™; eth=ethanol; Raman spectra of COINs (C) were overlaid with spectra obtained from SERS (S); showing CON spectra can have different major peaks compared with respective SERS.

FIG. 10 shows absorption spectra of Raman labels. 25 μM 8-aza-adenine (AA) and 5 μM N-benzoyl adenine (BA) were used to make COINs, respectively; after COIN synthesis, the COIN solutions were filtered through 300 kDa filter (Pall Life Sciences, through VWR) units by centrifugation (1000×g for 5 min) and the clear solutions were used for absorption measurement; also shown were absorption spectra of 25 μM AA and 5 μM BA and 1 mM Na₃Citrate; the data suggested that the free Raman label molecules were depleted from the solutions.

FIGS. 11A and B show COIN signatures obtained in multiplex analysis (continued from FIG. 7). COINs were made by the oven incubation procedure described above with mixtures of 2 or 3 Raman labels at concentrations from 2.5 to 20 μM, depending on signatures desired. The 3 Raman labels were 8-aza-adenine (AA), 9-aminoacridine (AN), methylene blue (MB). The main peak positions are indicated by arrows; the peak heights (in arbitrary unit) were normalized to respective maximums; spectra are offset by 1 unit from each other. FIG. 11A shows signatures of COIN made with 2 Raman labels (AA and MB) at concentrations so that indicated relative peak heights were obtained: AA=MB (HH), AA>MA (HL) and AA<MB (LH). FIG. 11B shows Raman signatures of COINs made from mixtures of the 3 Raman labels at concentrations that produced signatures as indicated: HHL means high peak intensities for AA (H) and AN (H) and low peak intensity for MB (L); HLH means high peak intensity for AA (H) and low peak intensities for AN (L) and high peak intensity for MB (H). Other features could be revealed by computer analysis.

FIG. 12 is a schematic of exemplary COIN-containing microspheres.

FIG. 13 is a flow chart illustrating a method for producing COIN-containing microspheres (the inclusion method).

FIG. 14 illustrates an alternative method for producing COIN-containing microspheres (the soak-in method).

FIG. 15 illustrates another alternative method for producing COIN-containing microspheres (the build-in method).

FIG. 16 illustrates another alternative method for producing COIN-containing microspheres (the build-out method).

### DETAILED DESCRIPTION OF THE INVENTION

Embodiments of the invention provide composite organic inorganic nanoclusters (COINs) that are comprised of several fused or aggregated metal particles that form a metallic cluster containing Raman-active organic compounds adsorbed to the surfaces of the aggregated particles and in the junctions where primary metal particles meet. In general, COINs can be synthesized from a wide range of organic compounds to produce enhanced Raman signals and distinguishable Raman signatures. Because a wide range of COINs can be synthesized, in embodiments of the invention COINs are used as a coding system for multiplex detection of bioanalytes.

In general, the particles according to the present invention are less than about 1 μm size and are formed by particle growth in the presence of organic compounds. The preparation of such nanoclusters takes advantage of the ability of metals to adsorb organic compounds. Indeed, since Raman-active organic compounds are adsorbed onto the metal particles during formation of the metallic colloids, many Raman-active organic compounds can be incorporated into a nanoparticle without requiring special attachment chemistry.

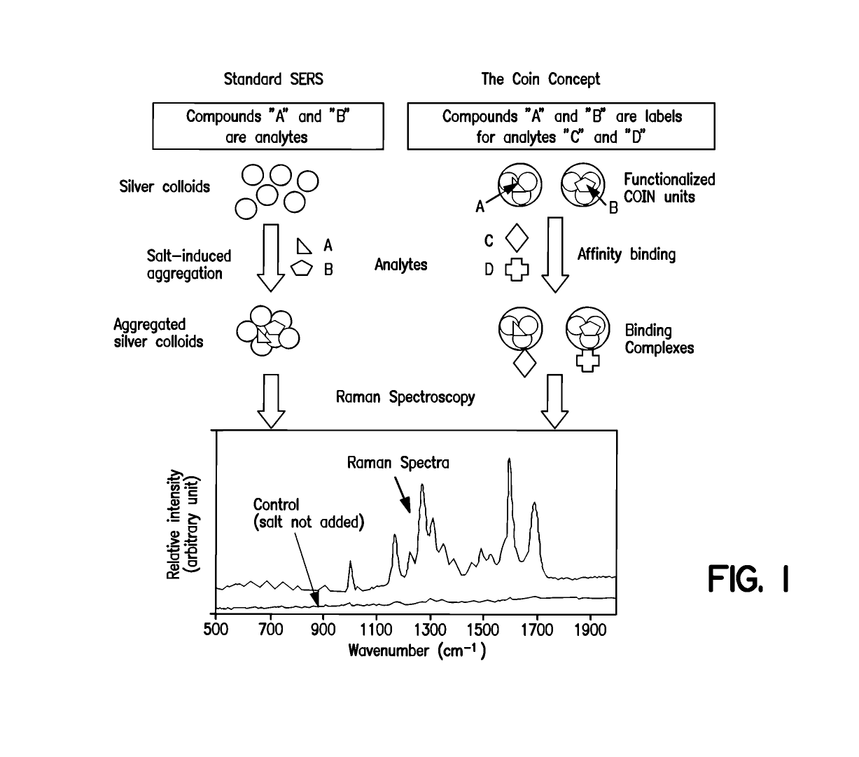

In a typical SERS measurement, an analyte is detected by depositing on or co-aggregating metal atoms or colloids with an analyte. As illustrated in FIG. 1A, standard SERS can be used as an amplification step to detect target molecules “A” and “B” according to their Raman signatures by adsorbing the target analytes, “A” and “B,” onto silver particles. The spectra of FIG. 1C show that SERS signal obtained after colloid aggregation induced by salts was at least 10 times stronger than that without salt addition, in which the hardly detectable signals resulted from label-induced colloid aggregation.

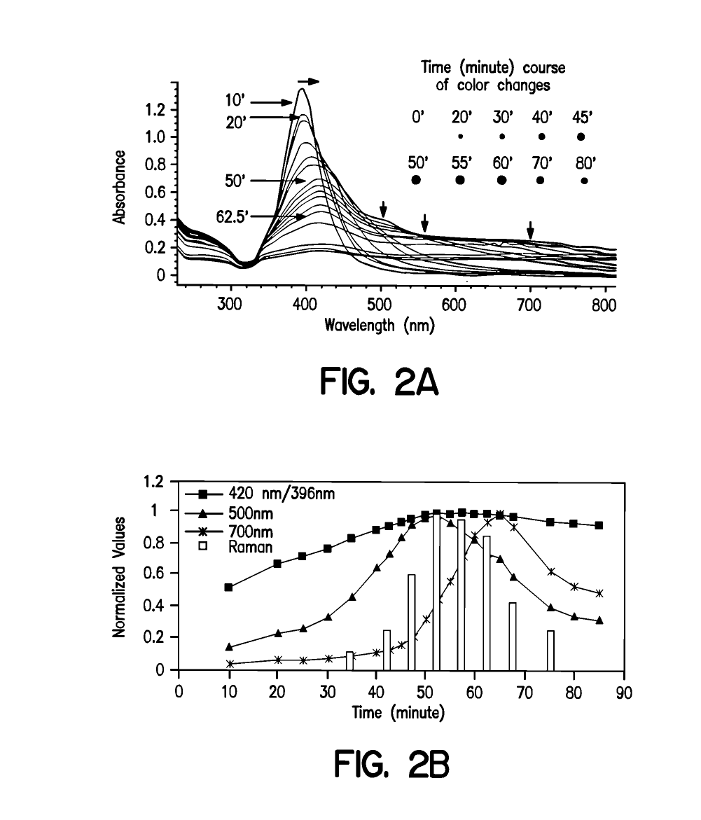

It was discovered that organic compounds could be adsorbed on metal colloids and cause metal colloid aggregation. Additionally, it was found that the aggregated metal colloids fused at elevated temperatures. Organic Raman labels can be incorporated into the coalescing metal particles to produce composite organic-inorganic nanoclusters (COINs) with intrinsic SERS activities. For example, we synthesized silver seed colloids of about 12 nm in diameter and mixed the silver colloids with an organic Raman label (e.g., 20 μM 8-aza-adenine) and then generated additional metal silver from AgNO₃ by heating the solution in the presence of a reductant. The solution color changed from yellow to orange, then brown and finally blue. The color changes were quantified by absorbance measurement (FIG. 2A). The main absorbance peak red-shifted from 395 nm in the first 50 min. and then remained around 420 nm. At the same time, a small shoulder peak at 500 nm appeared (FIG. 2B). Afterward, the absorption at higher wavelengths (i.e., 700 nm) increased until the 62.5 min. time point. During the 12.5 min. time period, SERS activity reached maximum (FIG. 2B). Since SERS activity peaked after the completion of main peak transition and before the start of silver aggregate sedimentation (before 700 nm peak decreased), we concluded that SERS-active COIN formation has two phases: a particle enlargement (fusion) phase and a subsequent particle clustering phase. The two phase process is supported by electron microscopy studies. When a silver seed suspension was heated to 100°° C. for 40 min. in the absence of an organic Raman label, the solution maintained a light orange color and the majority of the silver particles remained less than 10 nm. A Raman label was added into a silver seed solution and the solution was heated to develop an orange color. At this point, SERS activity was not detectable, and most of the small silver colloids turned into relatively large ones of greater than10 nm. After an extended heating, a brownish color developed which was associated with strong Raman activity. At this stage, in this example, particle clusters comprising two or more primary particles became apparent. Scanning electron microscopy (SEM) analysis indicated that the SERS-active particles in this example were aggregates of about 100 nm comprising primary particles of about 20-30 nm.

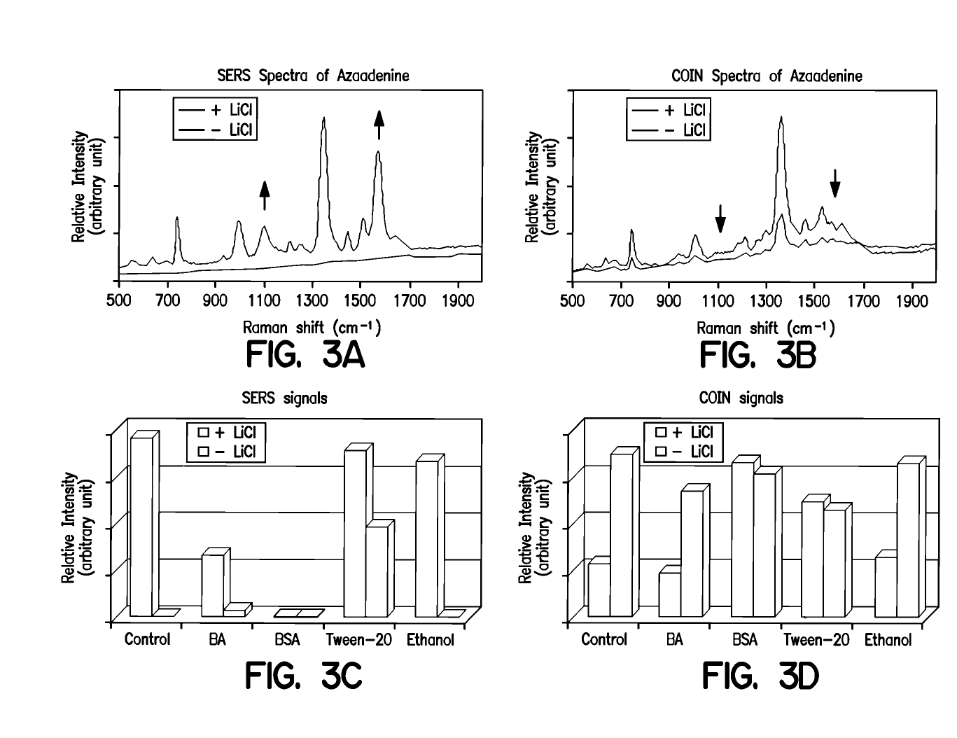

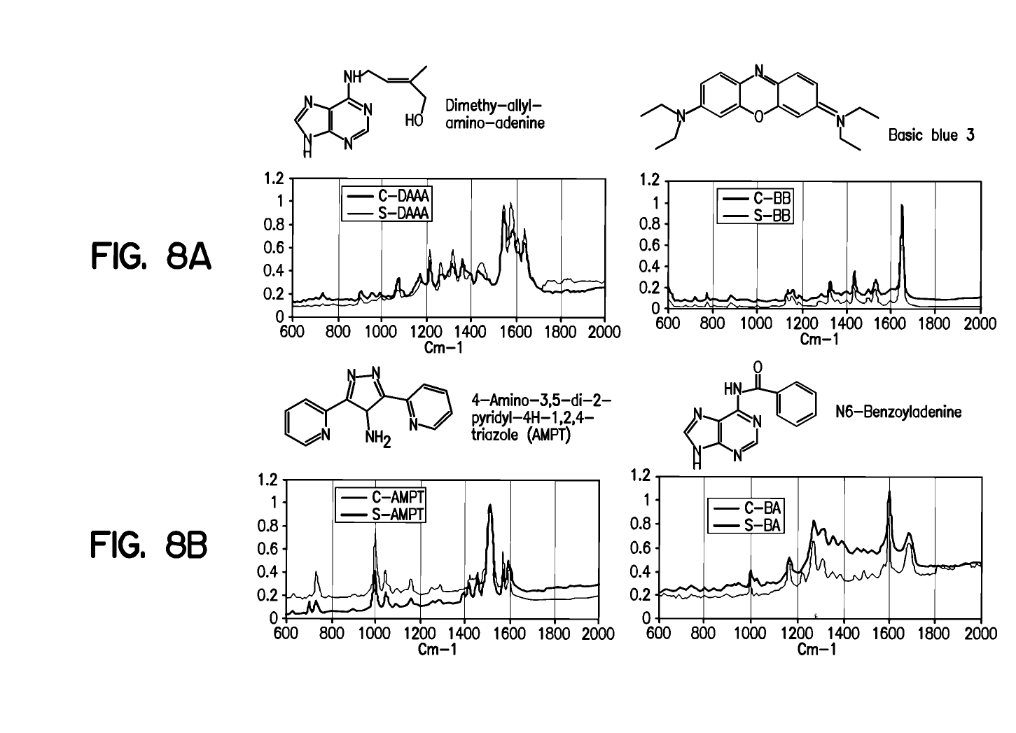

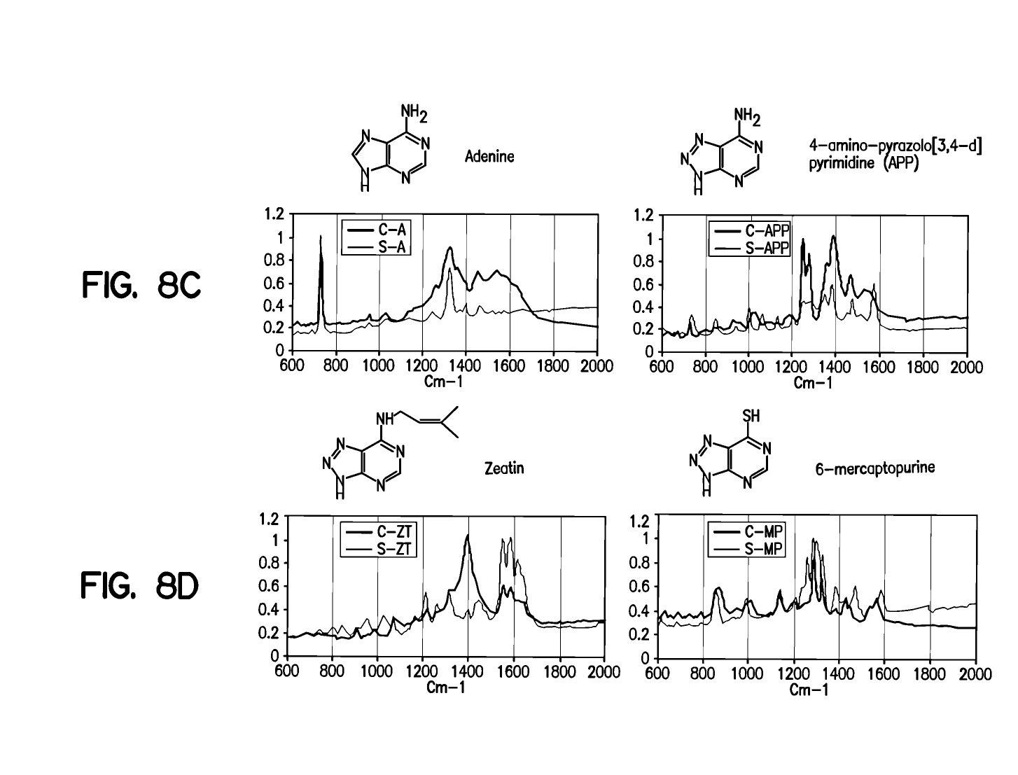

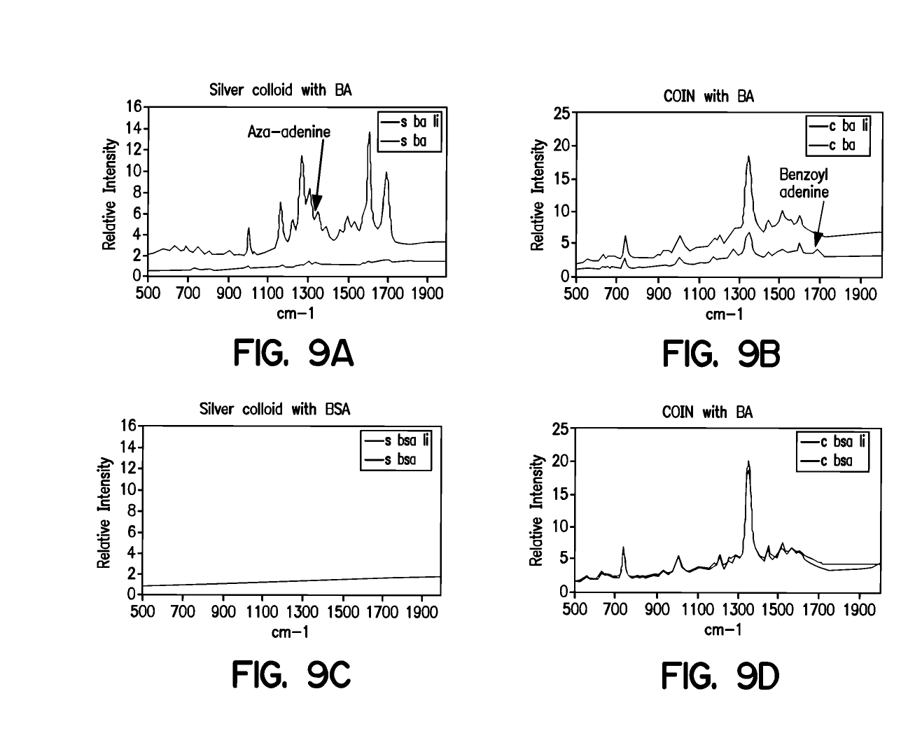

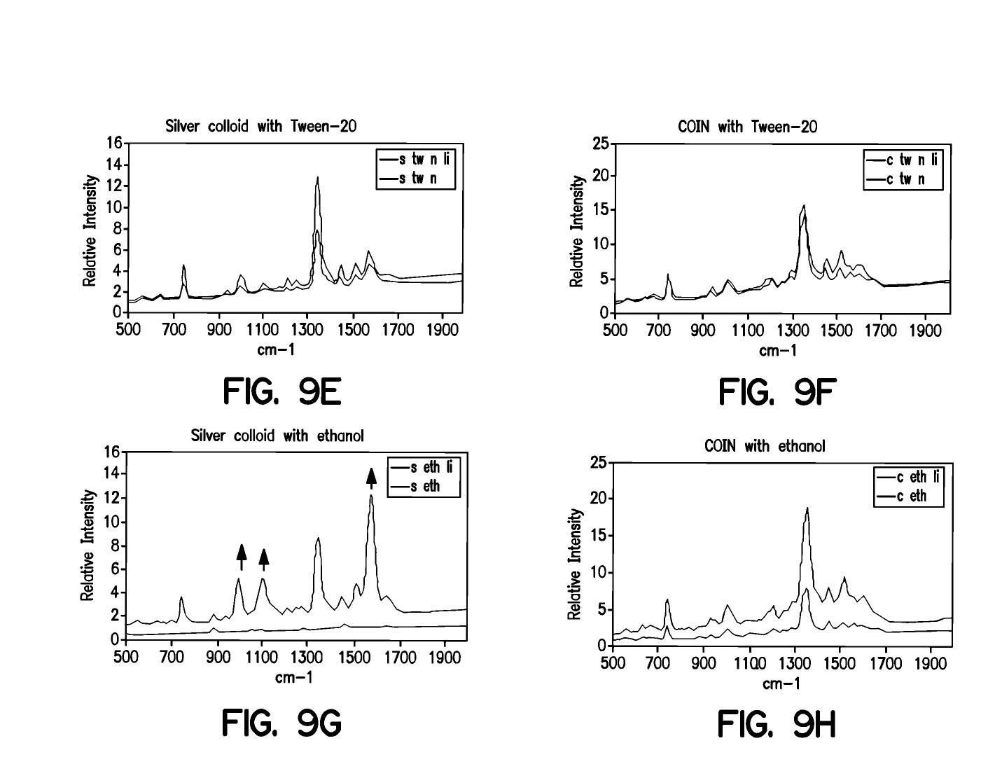

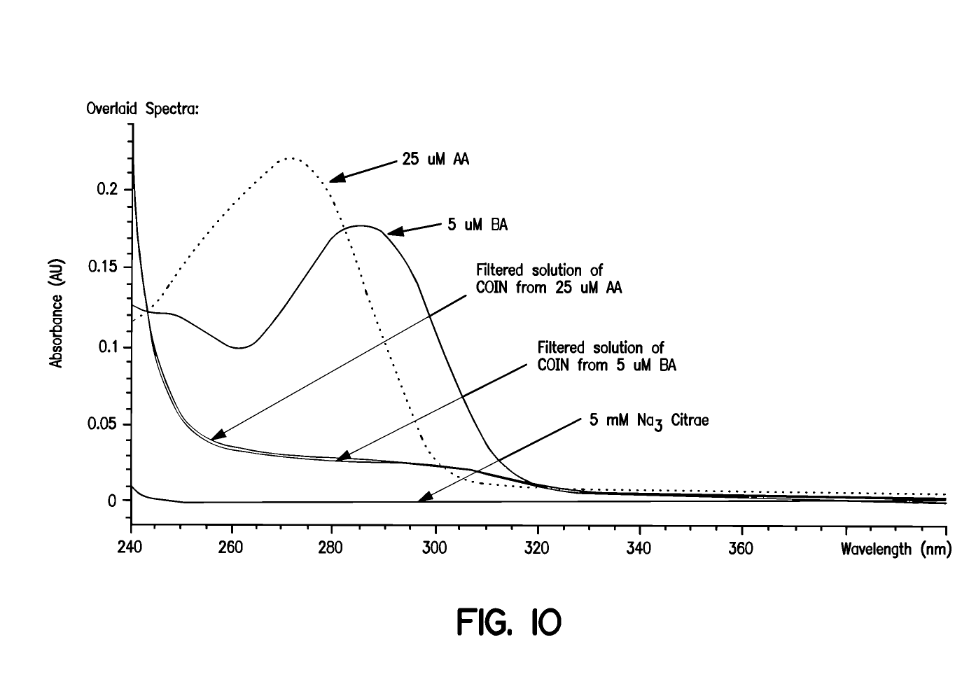

COINs generate intrinsic SERS signals. We compared SERS-activities of COINs with data from typical SERS reactions in the presence of various test agents (FIG. 3A to 3D). Typical SERS reactions require addition of salt to induce aggregation of nanoparticles for strong SERS activity. FIG. 3A shows a typical Raman spectrum when a Raman label (8-aza-adenine) was mixed with silver colloids and a monovalent salt (+LiCl). When the salt was omitted from the reaction (−LiCl), SERS signal was not detectable. By contrast, a strong Raman signal was detected from a COIN sample with no salt added (FIG. 3B) and when salt was included the Raman signal was greatly reduced, possibly due to increased aggregation and sedimentation of the COIN particles. Compared with the typical SERS spectrum, the peaks at 1100 cm⁻¹ and 1570 cm⁻¹ disappeared almost completely from the COIN spectrum. Spectral differences were also observed from other Raman labels that had been tested (see examples in FIG. 8). For example, COIN particles had negligible Raman enhancement activity for the test Raman labels (10 μM N-benzoyl adenine, see FIGS. 9A-B). It was also observed that SERS signals were completely suppressed by 0.3% bovine serum albumin (BSA). By contrast, signals of COIN did not change significantly in the presence of added BSA, regardless of the presence or absence of salt. Tween-20®, a nonionic surfactant commonly used in biochemical reactions, appeared to inhibit salt-induced aggregation but cause a low degree of colloid aggregation as observed in separate experiments. It was interesting to find that SERS reaction in the presence of 30% ethanol (plus salt) enhanced the peak height at 1550 cm⁻¹ compared with ethanol free reactions (FIG. 9G). On the other hand, COIN signals were equivalent to COINs in water in terms of spectra and relative peak intensities (FIG. 3D and FIG. 9H). These functional analyses show clearly that COINs have distinct chemical and physical properties from salt-induced colloid aggregates as used in typical SERS reactions.

In additional embodiments, there are provided methods for producing composite organic-inorganic nanoclusters. Such methods can be performed, for example, by reducing metal ions in the presence of a Raman-active organic compound under conditions suitable to form a metallic colloid, thereby producing a cluster of several fused or aggregated metal particles with the Raman-active organic compound adsorbed on the metal particles and/or in the junctions of the metal particles.

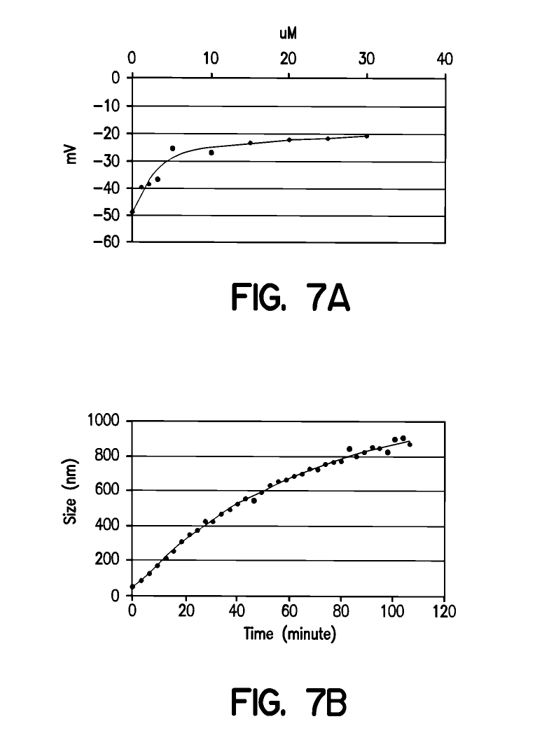

The nanoclusters according to the invention can be prepared by a physico-chemical process called Organic Compound Assisted-Metal Fusion (OCAMF). Organic compounds can be adsorbed on metal colloids and cause aggregation by changing the surface zeta potentials of the particles (FIGS. 7A-B) and it was found that the aggregated metal colloids fused at elevated temperature. Organic Raman labels can be incorporated into the coalescing metal particles to form stable clusters and produce intrinsically enhanced Raman scattering signals. The interaction between the organic Raman label molecules and the metal colloids has mutual benefits. Besides serving as signal sources, the organic molecules promote and stabilize metal particle association that is in favor of EME of SERS. On the other hand, the metal particles provide spaces to hold and stabilize Raman label molecules, especially in the cluster junctions. These composite organic-inorganic nanoclusters (COINs) may be used as reporters for molecular probes. This concept is illustrated in FIG. 1B, in which 2 types of COINs can be made from compounds “A” and “B” and then functionalized with specific affinity probes to detect analytes “C” and “D” through attachment of the probe to the analyte of interest.

Using the OCAMF-based COIN synthesis chemistry, it is possible to generate a large number of different COIN signatures by mixing a limited number of Raman labels. Thus COINs are suitable for use in multiplex assays. In a simplified scenario, the Raman spectrum of a sample labeled with COINs can be characterized by three parameters:

(a) peak position (designated as L), which depends on the chemical structure of Raman labels used and the umber of available labels,

(b) peak number (designated as M), which depends on the number of labels used together in a single COIN, and

(c) peak height (designated as i), which depends on the ranges of relative peak intensity.

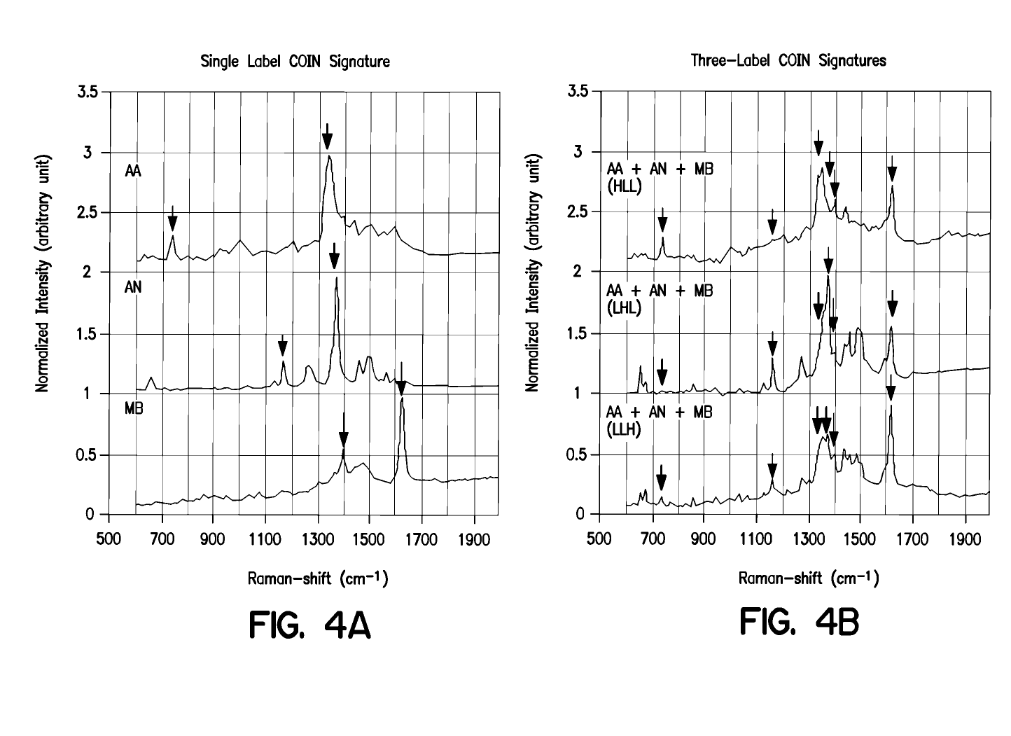

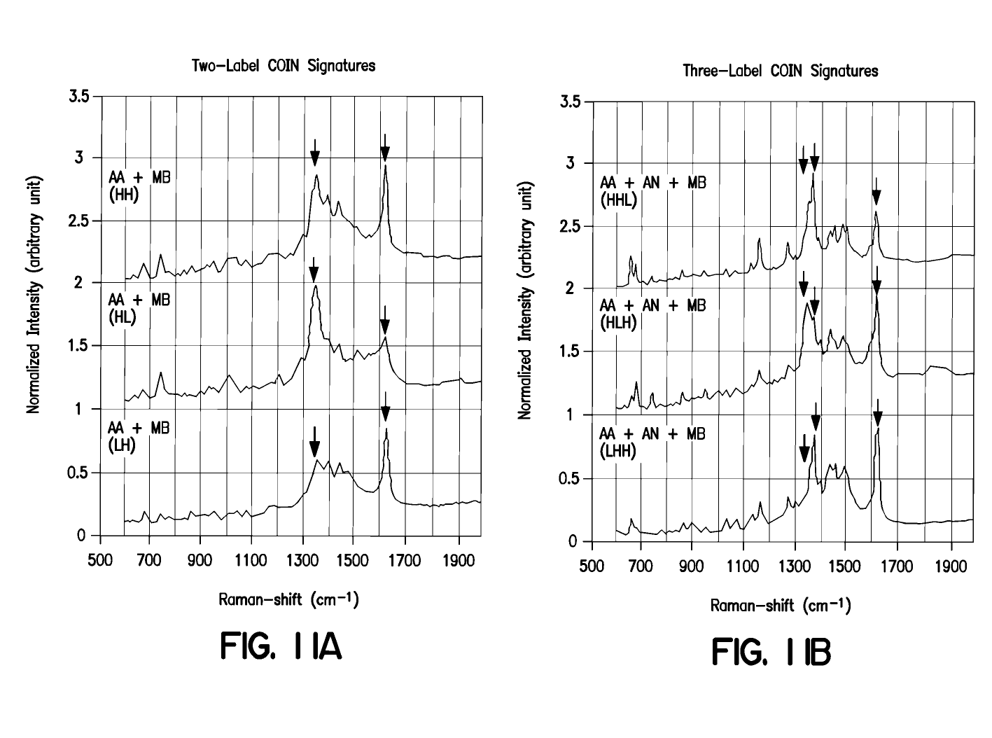

The total number of possible Raman signatures (designated as T) can be calculated from the following equation:

$$T = {\sum }_{k = 1}^{M} \frac{L !}{( L - k ) ! k !} P ( i , k )$$

where P(i, k)=i^{k}−i+1, being the intensity multiplier which represents the number of distinct Raman spectra that can be generated by combining k (k=1 to M) labels for a given i value. To demonstrate that multiple labels can be mixed to make COINs, we tested the combinations of 3 Raman labels for COIN synthesis (L=3, M=3, and i=2). As shown in FIG. 4 (also see FIG. 11), the results for 1 label, 2 labels and 3 labels were all as expected. These spectral signatures demonstrated that closely positioned peaks (15 cm⁻¹ between AA and AN) could be resolved visually. Theoretically, over a million of COIN signatures can be made within the Raman shift range of 500-2000 cm⁻¹ by incorporating multiple organic molecules into COINs as Raman labels using the OCAMF-based COIN synthesis chemistry.

As used herein, the term organic compound refers to any hydrocarbon molecule containing at least one aromatic ring and at least one nitrogen atom. Organic compounds may also contain atoms such as O, S, P, and the like. As used herein, Raman-active organic compound refers to an organic molecule that produces a unique SERS signature in response to excitation by a laser. A variety of organic compounds, both Raman-active and non-Raman active, are contemplated for use as components in nanoclusters. In certain embodiments, Raman-active organic compounds are polycyclic aromatic or heteroaromatic compounds. Typically the Raman-active compound has a molecular weight less than about 500 Daltons.

In several non-limiting examples, a variety of organic Raman labels, as shown in Table 1, were used for COIN synthesis. The compounds tested can be divided into several classes: (a) colorless and non-fluorescent (e.g., 8-aza-adenine), (b) colored dyes (e.g., methylene blue), (c) fluorescent dyes (e.g., 9-aminoacridine), and (d) thiol compounds (e.g., 6-mercaptopurine). All of the compounds were soluble in aqueous solutions at less than 1 mM. Note that the Raman shift peaks from COINs do not necessarily match those of SERS. In testing, over 40 organic compounds showed positive signals when incorporated into COIN (Table 1 and FIG. 8), of which fluorescent dyes gave the strongest COIN signals.

TABLE 1

| No. | Abbreviation | Name | Structure |
|---|---|---|---|
| 1 | AAD (AA) | 8-Aza-Adenine | 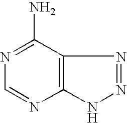 |
| 2 | BZA (BA) | N-Benzoyladenine | 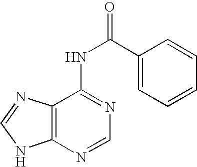 |
| 3 | MBI | 2-Mercapto- benzimidazole | 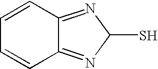 |
| 4 | APP | 4-Amino- pyrazolo[3,4- d]pyrimidine | 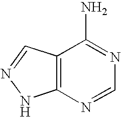 |
| 5 | ZEN | Zeatin | 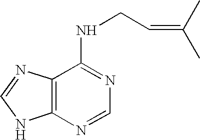 |
| 6 | MBL (MB) | Methylene Blue | 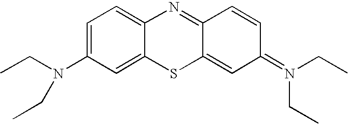 |
| 7 | AMA (AN, AM) | 9-Amino-acridine | 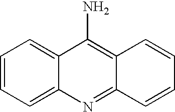 |
| 8 | EBR | Ethidium Bromide | 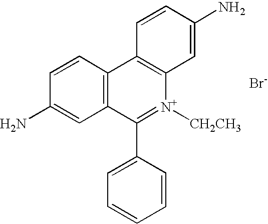 |
| 9 | BMB | Bismarck Brown Y | 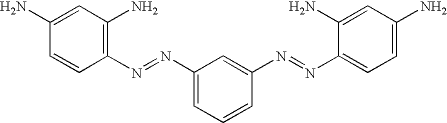 |
| 10 | NBA | N-Benzyl- aminopurine | 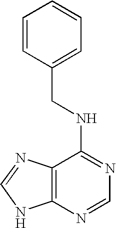 |
| 11 | THN | Thionin acetate | 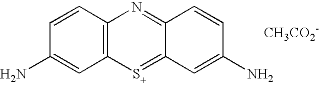 |
| 12 | DAH | 3,6-Diaminoacridine | 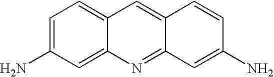 |
| 13 | CYP | 6-Cyanopurine | 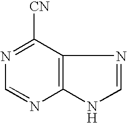 |
| 14 | AIC | 4-Amino-5-imidazole- carboxamide hydrochloride | 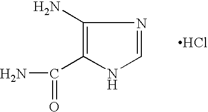 |
| 15 | DII | 1,3- Diiminoisoindoline | 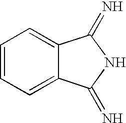 |
| 16 | R6G | Rhodamine 6G | 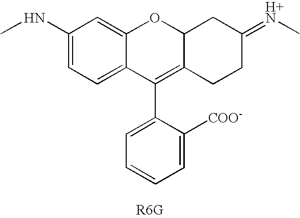 |
| 17 | CRV | Crystal Violet | 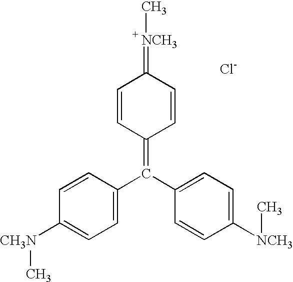 |
| 18 | BFU | Basic Fuchsin | 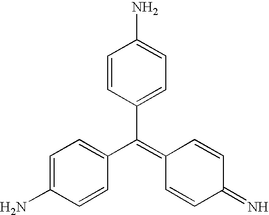 |
| 19 | ANB | Aniline Blue diammonium salt | 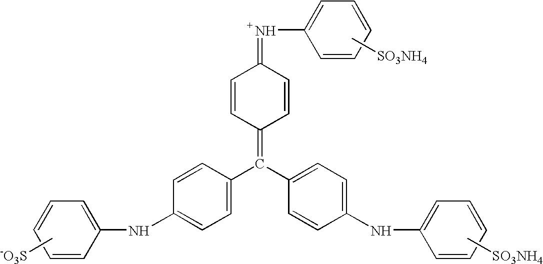 |
| 20 | ACA | N-[(3- (Anilinomethylene)- 2-chloro-1- cyclohexen-1- yl)methylene]aniline monohydrochloride | 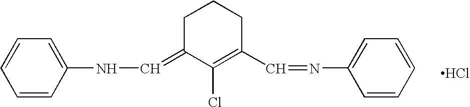 |
| 21 | ATT | O-(7- Azabenzotriazol-1- yl)-N,N,N′,N′- tetramethyluronium hexafluorophosphate | 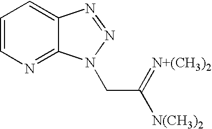 |
| 22 | AMF | 9-Aminofluorene hydrochloride | 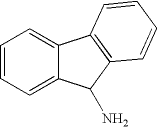 |
| 23 | BBL | Basic Blue | 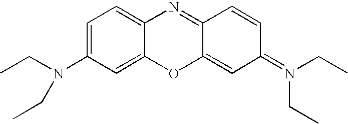 |
| 24 | DDA | 1,8-Diamino-4,5- dihydroxyanthraquin one | 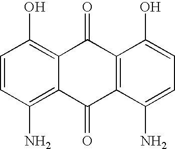 |
| 25 | PFV | Proflavine hemisulfate salt hydrate | 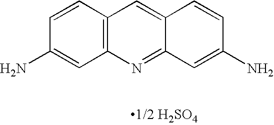 |
| 26 | APT | 2-Amino-1,1,3- propenetricarbonitrile | 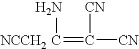 |
| 27 | VRA | Variamine Blue RT Salt | 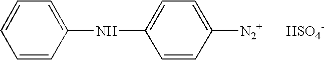 |
| 28 | TAP | 4,5,6- Triaminopyrimidine sulfate salt | 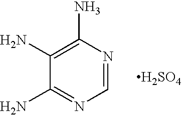 |
| 29 | ABZ | 2-Amino- benzothiazole | 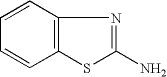 |
| 30 | MEL | Melamine | 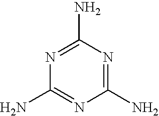 |
| 31 | PPN | 3- (3-Pyridylmethylamino)- propionitrile | 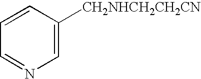 |
| 32 | SSD | Silver(I) sulfadiazine | 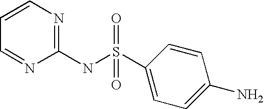 |
| 33 | AFL | Acriflavine | 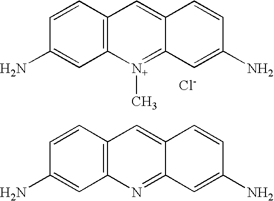 |
| 34 | AMPT | 4-Amino6- Mercaptopyrazolo[3,4- d]pyrimidine | 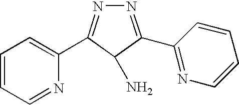 |
| 35 | APU | 2-Am-Purine | 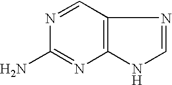 |
| 36 | ATH | Adenine Thiol | 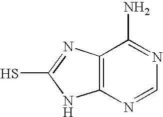 |
| 37 | FAD | F-Adenine | 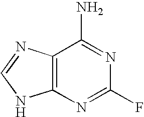 |
| 38 | MCP | 6-Mercaptopurine | 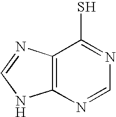 |
| 39 | AMP | 4-Amino-6- mercaptopyrazolo[3,4- d]pyrimidine | 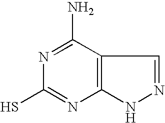 |
| 41 | R110 | Rhodamine 110 | 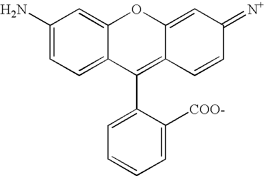 |
| 42 | ADN | Adenine | 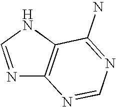 |
| 43 | AMB | 5-amino-2- mercaptobenzimidazole | 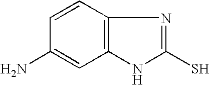 |

In addition, it is understood that these Raman-active compounds can include fluorescent compounds or non-fluorescent compounds. Exemplary Raman-active organic compounds include, but are not limited to, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, 9-amino-acridine, and the like.

Additional, non-limiting examples of Raman-active organic compounds include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescine, 5-carboxy rhodamine, 6-carboxyrhodamine, 6-carboxytetramethyl amino phthalocyanines, azomethines, cyanines, xanthines, succinylfluoresceins, aminoacridine, and the like. These and other Raman-active organic compounds may be obtained from commercial sources (e.g., Molecular Probes, Eugene, Oreg.).

When fluorescent compounds are incorporated into nanoclusters described herein, the compounds include, but are not limited to, dyes, intrinsically fluorescent proteins, lanthanide phosphors, and the like. Dyes include, for example, rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃-coumarin-3-acetate, 7-NH₂-4CH₃-coumarin-3-acetate (AMCA), monobromobimane, pyrene trisulfonates, such as Cascade Blue, and monobromotrimethyl-ammoniobimane.

The OCAMF chemistry allows the incorporation of a wide range Raman labels into metal colloids to produce numerous types of COINs. The simple one-step chemical procedure makes it possible to do parallel synthesis of a large number of COINs with different Raman signatures in a matter of hours by mixing several organic Raman-active compounds in different ratios.

The metal nanoparticles used for COIN synthesis can vary in size, but are chosen to be smaller than the size of the desired resulting COINs. For some applications, for example, in the oven and reflux synthesis methods, silver particles ranging in average diameter from about 3 to about 12 nm were used to form silver COINs and gold nanoparticles ranging from about 13 to about 15 nm were used to make gold COINs. In another application, for example, silver particles having a broad size distribution of about 10 to about 80 nm were used in a cold synthesis method. Typical metals contemplated for use in formation of nanoclusters include, for example, silver, gold, platinum, copper, aluminum, and the like. Additionally, multi-metal nanoparticles may be used, such as, for example, silver nanoparticles having gold cores.

Typically, for applications such as analyte detection, COINs range in average diameter from about 20 nm to about 200 nm, and more preferably COINs range in average diameter from about 30 to about 200 nm, and more preferably from about 40 to about 200 nm, more preferably from about 50 to about 200 nm, and more preferably about 50 to about 150 nm.

In certain embodiments of the invention, the metal particles used are metal colloids. As used herein, the term colloid refers to a category of complex fluids consisting of nanometer-sized particles suspended in a liquid, usually an aqueous solution. During metal colloid formation or growth in the presence of organic molecules in the liquid, the organic molecules are adsorbed on the primary metal particles suspended in the liquid and/or in interstices between primary metal particles. Typical metals contemplated for use in formation of nanoclusters from metal colloids include, for example, silver, gold, platinum, copper, aluminum, and the like. A typical average size range for the metal particles in the colloids used in the invention methods and compositions is from about 3 nm to about 15 nm.

In general, COINs can be prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This type of nanoparticle is a cluster or aggregate of several primary metal particles with the Raman-active organic compounds adsorbed on the surfaces of the metal particles and trapped in the junctions between the primary metal particles. The COINs are not usually spherical and often include grooves and protuberances. COINs can be isolated by membrane filtration and COINs of different sizes can be enriched by centrifugation.

In further embodiments of the invention, the nanoclusters include a second metal different from the first metal, wherein the second metal forms a layer overlying the surface of the nanocluster. To prepare this type of nanocluster, COINs are placed in an aqueous solution containing suitable second metal cations and a reducing agent. The components of the solution are then subject to conditions that reduce the second metallic cations, thereby forming a metallic layer overlying the surface of the nanocluster. In certain embodiments, the second metal layer includes metals, such as, for example, silver, gold, platinum, aluminum, copper, zinc, iron, and the like. This type of nanocluster can be isolated and or enriched in the same manner as single-metal COINs.

In certain embodiments, the metallic layer overlying the surface of the nanocluster is referred to as a protection layer. A protection layer can contribute to aqueous stability of the nanoclusters. As an alternative to metallic protection layers or in addition to metallic protection layers, COINs can be coated with a layer of silica. If the COINs have already been coated with a metallic layer, such as for example, gold, a silica layer can be attached to the gold layer by vitreophilization of the COINs with, for example, 3-aminopropyltrimethoxysilane (APTMS). Silica deposition is initiated from a supersaturated silica solution, followed by growth of a silica layer by dropwise addition of ammonia and tetraethyl orthosilicate (TEOS). The silica-coated COINs are readily functionalized using standard silica chemistry.

In certain other embodiments, COINs can include an organic layer overlying the metal surface or the silica layer. In some embodiments, these types of nanoparticles are prepared by adsorbing or covalently attaching organic compounds to the surface of the COINs. Covalent attachment of an organic layer to the metal surface can be achieved in a variety ways well known to those skilled in the art, such as for example, through thiol-metal bonds. In alternative embodiments, the organic molecules can be crosslinked to form a molecular network.

An organic layer can also be used to provide colloidal stability and functional groups for further derivatization. The organic layer is optionally crosslinked to form a solid coating. An exemplary organic layer is produced by adsorption of an octylamine modified polyacrylic acid onto COINs, the adsorption being facilitated by the positively charged amine groups. The carboxylic groups of the polymer are then crosslinked with a suitable agent such as lysine, (1,6)-diaminoheptane, and the like. Unreacted carboxylic groups can be used for further derivation. Other functional groups can be also introduced through the modified polyacrylic backbones.

Furthermore, the metal and organic coatings can be overlaid in various combinations to provide desired properties of coated COINs. For example, COINs may be first coated with a gold layer to seal the more reactive silver before applying the adsorption layer, silica or solid organic coatings. Even if the outer layer is porous, the inner gold layer can prevent COINs from chemical attack by reagents used in different applications. Another example is to apply an adsorption layer on silica or gold layer to provide additional colloidal stability.

In certain other embodiments, the metal particles used in COINs can include magnetic materials, such as, for example, iron oxides, and the like. Magnetic COINs can be handled without centrifugation using commonly available magnetic particle handling systems. Indeed, magnetism can be used as a mechanism for separating COIN particles tagged with particular biological probes.

In yet other embodiments, there are provided methods for detecting an analyte in a sample. Such methods can be performed, for example, by contacting a sample containing an analyte with COINs having an attached probe, wherein the probe binds to the analyte, separating any COIN-analyte complexes from any uncomplexed COINs, and detecting SERS signals from the nanocluster, wherein the SERS signals are indicative of the presence of an analyte.

In further embodiments, there are provided methods for identifying analytes in a sample using a set of Raman-active metallic nanoclusters with each member of the set having a Raman signature unique to the set. Such methods can be performed, for example, by contacting a sample suspected of containing the analytes with a plurality of the nanoclusters; detecting SERS signals in multiplex fashion upon contacting the sample with the nanoclusters; and associating the SERS signals from the nanoclusters with the identity of analytes to which the nanoclusters attach.

In other embodiments, the invention provides methods for distinguishing biological analytes in a sample by contacting a sample comprising a plurality of biological analytes with a set of Raman-active metallic clusters having an average diameter of about 50 nm to about 200 nm with each member of the set having a Raman signature unique to the set produced by at least one Raman active organic compound incorporated therein and a probe that binds specifically to a known biological analyte under conditions suitable to allow specific binding of probes to analytes present in the sample to form complexes. The bound clusters are separated and Raman signatures emitted by the organic Raman active compounds in the bound complexes are detected in a multiplex fashion. Each Raman signature indicates the presence of the known biological analyte in the sample.

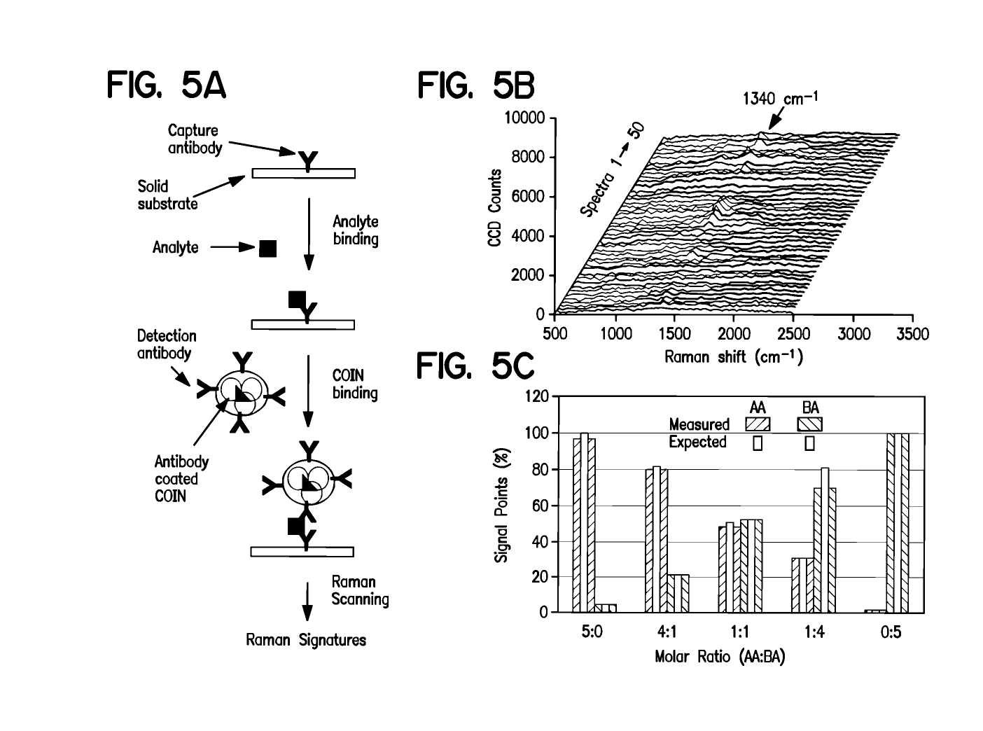

COINs can be used as tags for bio-analyte detection and, in one example, we used an assay scheme similar to a standard sandwich immuno assay (FIG. 5A); except that the signal amplification step after specific binding that is necessary in sandwich immunoassays using other labels is not needed when COINs are used as the analyte tags (FIG. 5A). The protein interleukin-2 (IL-2) was attached to surfaces that were precoated with anti-IL-2 capture antibody so that the maximum average IL-2 molecule density was less than 1 molecule per laser beam cross section area (0.77 molecules per 12 micron², 1.3 yoctomole within the laser beam) and anti-IL-2 antibody-coated COINs were used to detect immobilized IL-2 molecules. As shown in FIG. 5B, an average of 28% spectra were observed that had the desired IL-2 signature, suggesting a 36% detection rate for all applied analyte molecules. This detection rate could be possible, under the experimental conditions, only when each data collection area had, on average, less than 10 analyte molecules, considering possible incomplete binding in the sandwich assay and the possible presence of inactive COINs.

Capture substrates were prepared with mixed antibodies against IL-2 and IL-8. Similarly, two sets of COINs (with signatures for AA and BA, respectively) were prepared with detection antibodies that bind specifically to the two analytes. When different ratios of the two analytes were used, positive COIN signals were detected at ratios that matched well with the expected values based on the known ratios of analytes used (FIG. 5C).

For use in the detection of analytes a probe can be attached to the COIN. In certain embodiments, exemplary probes are antibodies, antigens, polynucleotides, oligonucleotides, receptors, ligands, and the like. The term polynucleotide is used broadly herein to mean a sequence of deoxyribonucleotides or ribonucleotides that are linked together by a phosphodiester bond. For convenience, the term oligonucleotide is used herein to refer to a polynucleotide that is used as a primer or a probe. Generally, an oligonucleotide useful as a probe or primer that selectively hybridizes to a selected nucleotide sequence is at least about 10 nucleotides in length, usually at least about 15 nucleotides in length, and for example between about 15 and about 50 nucleotides in length. Polynucleotide probes are particularly useful for detecting complementary polynucleotides in a biological sample and can also be used for DNA sequencing by pairing a known polynucleotide probe with a known Raman-active signal made up of a combination of Raman-active organic compounds as described herein.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides.

The covalent bond linking the nucleotides of a polynucleotide generally is a phosphodiester bond. However, the covalent bond also can be any of numerous other bonds, including a thiodiester bond, a phosphorothioate bond, a peptide-like amide bond or any other bond known to those in the art as useful for linking nucleotides to produce synthetic polynucleotides. The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

As used herein, the term selective hybridization or selectively hybridize, refers to hybridization under moderately stringent or highly stringent conditions such that a nucleotide sequence preferentially associates with a selected nucleotide sequence over unrelated nucleotide sequences to a large enough extent to be useful in identifying the selected nucleotide sequence. It will be recognized that some amount of non-specific hybridization can occur, but is acceptable provided that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar or homologous nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10-15 minutes each, in the order listed above, repeating any or all of the steps listed. However, as mentioned above, optimal conditions will vary, depending on the particular hybridization reaction involved, and can be determined empirically.

In some embodiments, the organic layer can include an antibody probe. As used herein, the term antibody is used in its broadest sense to include polyclonal and monoclonal antibodies, as well as antigen binding fragments of such antibodies. An antibody useful in a method of the invention, or an antigen binding fragment thereof, is characterized, for example, by having specific binding activity for an epitope of an analyte.

An antibody is associated with the nanoclusters in certain aspects of the invention. The antibody, for example, includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains. These and other methods of making, for example, chimeric, humanized, CDR-grafted, single chain, and bifunctional antibodies are well known to those skilled in the art.

The term binds specifically or specific binding activity, when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)2, Fd and Fv fragments of an antibody that retain specific binding activity for an epitope of an antigen, are included within the definition of an antibody.

In the context of the invention, the term ligand denotes a naturally occurring specific binding partner of a receptor, a synthetic specific-binding partner of a receptor, or an appropriate derivative of the natural or synthetic ligands. As one of skill in the art will recognize, a molecule (or macromolecular complex) can be both a receptor and a ligand. In general, the binding partner having a smaller molecular weight is referred to as the ligand and the binding partner having a greater molecular weight is referred to as a receptor.

In another embodiment, there are provided methods for detecting an analyte in a sample. Such methods can be performed, for example, by contacting a sample containing an analyte with a nanocluster including a probe, wherein the probe binds to the analyte; and detecting SERS signals emitted by the nanocluster, wherein the signals are indicative of the presence of an analyte. More commonly, the sample contains a pool of biological analytes an the sample is contacted with a set of COINs, as described herein, wherein each member of the set is provided with a probe that binds specifically to a known biological analyte and a different combination of Raman-active organic compounds are incorporated into members of the set to provide a unique Raman signature that can readily be correlated with the known analyte to which the probe will bind specifically.

By analyte is meant any molecule or compound. An analyte can be in the solid, liquid, gaseous or vapor phase. By gaseous or vapor phase analyte is meant a molecule or compound that is present, for example, in the headspace of a liquid, in ambient air, in a breath sample, in a gas, or as a contaminant in any of the foregoing. It will be recognized that the physical state of the gas or vapor phase can be changed by pressure, temperature as well as by affecting surface tension of a liquid by, for example, the presence of or addition of salts.

As indicated above, methods of the present invention, in certain aspects, detect binding of an analyte to a probe. The analyte can be comprised of a member of a specific binding pair (sbp) and may be a ligand, which is monovalent (monoepitopic) or polyvalent (polyepitopic), usually antigenic or haptenic, and is a single compound or plurality of compounds which share at least one common epitopic or determinant site. The analyte can be a part of a cell such as bacteria or a cell bearing a blood group antigen such as A, B, D, etc., or an HLA antigen or a microorganism, e.g., bacterium, fungus, protozoan, or virus. In certain aspects of the invention, the analyte is charged.

A member of a specific binding pair (sbp member) is one of two different molecules, having an area on the surface or in a cavity which specifically binds to and is thereby defined as complementary with a particular spatial and polar organization of the other molecule. The members of the specific binding pair are referred to as ligand and receptor (antiligand) or analyte and probe. Therefore, a probe is a molecule that specifically binds an analyte. These will usually be members of an immunological pair such as antigen-antibody, although other specific binding pairs such as biotin-avidin, hormones-hormone receptors, nucleic acid duplexes, IgG-protein A, polynucleotide pairs such as DNA-DNA, DNA-RNA, and the like are not immunological pairs but are included in the invention and the definition of sbp member.

Specific binding is the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide hybridization interactions, and so forth. Non-specific binding is non-covalent binding between molecules that is relatively independent of specific surface structures. Non-specific binding may result from several factors including hydrophobic interactions between molecules.

The nanoclusters of the present invention may be used to detect the presence of a particular target analyte, for example, a nucleic acid, oligonucleotide, protein, enzyme, antibody or antigen. The nanoclusters may also be used to screen bioactive agents, such as, for example, drug candidates, for binding to a particular target or to detect agents like pollutants. As discussed above, any analyte for which a probe moiety, such as a peptide, protein, oligonucleotide or aptamer, may be designed can be used in combination with the disclosed nanoclusters.

Ligand analytes include poly(amino acids), such as for example, polypeptides and proteins, polysaccharides, hormones, nucleic acids, and combinations thereof. Such combinations include components of bacteria, viruses, prions, cells, chromosomes, genes, mitochondria, nuclei, cell membranes and the like. Additional possible analytes include drugs, metabolites, pesticides, pollutants, and the like. Included among drugs of interest are the alkaloids. Among the alkaloids are morphine alkaloids, which includes morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites.

The term analyte further includes polynucleotide analytes such as those polynucleotides defined below. These include, for example, m-RNA, r-RNA, t-RNA, DNA, DNA-RNA duplexes. The term analyte also includes receptors that are polynucleotide binding agents, such as, for example, peptide nucleic acids (PNA), restriction enzymes, activators, repressors, nucleases, polymerases, histones, repair enzymes, chemotherapeutic agents, and the like.

The analyte may be a molecule found directly in a sample such as a body fluid from a host. The sample can be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

In general, probes can be attached to COINs through adsorption of the probe onto the COIN surface. Alternatively, COINs may be coupled with probes through biotin-avidin linkages. For example, avidin or streptavidin (or an analog thereof) can be adsorbed to the surface of the COIN and a biotin-modified probe contacted with the avidin or streptavidin-modified surface forming a biotin-avidin (or biotin-streptavidin) linkage. Optionally, avidin or streptavidin may be adsorbed in combination with another protein, such as BSA, and optionally be crosslinked. In addition, for COINs having a functional layer that includes a carboxylic acid or amine functional group, probes having a corresponding amine or carboxylic acid functional group can be attached through water-soluble carbodiimide coupling reagents, such as EDC (1-ethyl-3-(3-dimethyl aminopropyl)carbodiimide), which couples carboxylic acid functional groups with amine groups.

Nucleotides attached to a variety of functional groups may be commercially obtained (for example, from Molecular Probes, Eugene, Oreg.; Quiagen (Operon), Valencia, Calif.; and IDT (Integrated DNA Technologies), Coralville, Iowa) and incorporated into oligonucleotides or polynucleotides. Biotin-modified nucleotides are commercially available (for example, from Pierce Biotechnology, Rockford, Ill., or Panomics, Inc. Redwood City, Calif.) and modified nucleotides can be incorporated into nucleic acids during conventional amplification techniques. Oligonucleotides may be prepared using commercially available oligonucleotide synthesizers (for example, Applied Biosystems, Foster City, Calif.). Additionally, modified nucleotides may be synthesized using known reactions, such as for example, those disclosed in, Nelson, P., Sherman-Gold, R, and Leon, R, “A New and Versatile Reagent for Incorporating Multiple Primary Aliphatic Amines into Synthetic Oligonucleotides,” Nucleic Acids Res., 17:7179-7186 (1989) and Connolly, B., Rider, P. “Chemical Synthesis of Oligonucleotides Containing a Free Sulfhydryl Group and Subsequent Attachment of Thiol Specific Probes,” Nucleic Acids Res., 13:4485-4502 (1985). Alternatively, nucleotide precursors may be purchased containing various reactive groups, such as biotin, hydroxyl, sulfhydryl, amino, or carboxyl groups. After oligonucleotide synthesis, COINs may be attached using standard chemistries. Oligonucleotides of any desired sequence, with or without reactive groups for COIN attachment, may also be purchased from a wide variety of sources (for example, Midland Certified Reagents, Midland, Tex.).

Probes, such as polysaccharides, may also be attached to COINs, for example, through methods disclosed in Aslam, M. and Dent, A., Bioconjugation: Protein Coupling Techniques for the Biomedical Sciences, Grove's Dictionaries, Inc., 229, 254 (1998). Such methods include, but are not limited to, periodate oxidation coupling reactions and bis-succinimide ester coupling reactions.

The following paragraphs include further details regarding exemplary applications of COIN probes (composite organic-inorganic nanoclusters (COINs) that have an attached probe). It will be understood that numerous additional specific examples of applications that utilize COIN probes can be identified using the teachings of the present specification. One of skill in the art will recognize that many interactions between polypeptides and their target molecules can be detected using COIN labeled polypeptides. In one group of exemplary applications, COIN labeled antibodies (i.e. antibodies bound to a COIN) are used to detect interaction of the COIN labeled antibodies with antigens either in solution or on a solid support. It will be understood that such immunoassays can be performed using known methods such as, for example, ELISA assays, Western blotting, or protein arrays, utilizing the COIN-labeled antibody or COIN labeled secondary antibody, in place of a primary or secondary antibody labeled with an enzyme or a radioactive compound.

Another group of exemplary methods uses COIN probes to detect a target nucleic acid. Such a method is useful, for example, for detection of infectious agents within a clinical sample, detection of an amplification product derived from genomic DNA or RNA or message RNA, or detection of a gene (cDNA) insert within a clone. For certain methods aimed at detection of a target polynucleotide, an oligonucleotide probe is synthesized using methods known in the art. The oligonucleotide probe is then used to functionalize a COIN particle to produce a COIN labeled oligonucleotide probe. The COIN labeled oligonucleotide probe is used in a hybridization reaction to detect specific binding of the COIN labeled oligonucleotide probe to a target polynucleotide. For example, the COIN labeled oligonucleotide probe can be used in a Northern blot or a Southern blot reaction. Alternatively, the COIN labeled oligonucleotide probe can be applied to a reaction mixture that includes the target polynucleotide associated with a solid support, to capture the COIN labeled oligonucleotide probe. The captured COIN labeled oligonucleotide probe can then be detected using Raman spectroscopy, with or without first being released from the solid-support. Detection of the specific Raman label on the captured COIN labeled oligonucleotide probe, identifies the nucleotide sequence of the oligonucleotide probe, which in turn provides information regarding the nucleotide sequence of the target polynucleotide.

In another exemplary group of specific applications, a COIN labeled nucleotide is utilized to determine the nucleotide occurrence at a single base variation in a target polynucleotide. These applications include detection of “hot spot” point mutations and identification of the base at single nucleotide polymorphism (SNP) sites. For example, an oligonucleotide primer is prepared that hybridizes immediately adjacent to a polymorphic site. The primer, a target polynucleotide that includes the site of the single base variation, and a polymerase are included in an extension reaction mixture. The reaction mixture includes the four chain terminating triphosphates, each with a unique COIN label attached. The extension reaction then proceeds and, in the case of a homozygous SNP, only one of the four chain-terminating nucleotides is added to the end of the primer, thereby generating a COIN labeled elongated primer. The COIN label on the elongated primer is then detected using Raman spectroscopy. The identity of the label identifies the nucleotide added at the site of the single base variation, thereby identifying the nucleotide occurrence at the single base variation in the target polynucleotide.

In the methods of the invention, a sample includes a wide variety of analytes that can be analyzed using the nanoclusters described herein. For example, a sample can be an environmental sample and includes atmospheric air, ambient air, water, sludge, soil, and the like. In addition, a sample can be a biological sample, including, for example, a subject's breath, saliva, blood, urine, feces, various tissues, and the like.

Commercial applications for the invention methods employing the nanoclusters described herein include environmental toxicology and remediation, biomedicine, materials quality control, monitoring of food and agricultural products for the presence of pathogens, anesthetic detection, automobile oil or radiator fluid monitoring, breath alcohol analyzers, hazardous spill identification, explosives detection, fugitive emission identification, medical diagnostics, fish freshness, detection and classification of bacteria and microorganisms both in vitro and in vivo for biomedical uses and medical diagnostic uses, monitoring heavy industrial manufacturing, ambient air monitoring, worker protection, emissions control, product quality testing, leak detection and identification, oil/gas petrochemical applications, combustible gas detection, H₂S monitoring, hazardous leak detection and identification, emergency response and law enforcement applications, illegal substance detection and identification, arson investigation, enclosed space surveillance, utility and power applications, emissions monitoring, transformer fault detection, food/beverage/agriculture applications, freshness detection, fruit ripening control, fermentation process monitoring and control applications, flavor composition and identification, product quality and identification, refrigerant and fumigant detection, cosmetic/perfume/fragrance formulation, product quality testing, personal identification, chemical/plastics/pharmaceutical applications, leak detection, solvent recovery effectiveness, perimeter monitoring, product quality testing, hazardous waste site applications, fugitive emission detection and identification, leak detection and identification, perimeter monitoring, transportation, hazardous spill monitoring, refueling operations, shipping container inspection, diesel/gasoline/aviation fuel identification, building/residential natural gas detection, formaldehyde detection, smoke detection, fire detection, automatic ventilation control applications (cooking, smoking, etc.), air intake monitoring, hospital/medical anesthesia and sterilization gas detection, infectious disease detection and breath applications, body fluids analysis, pharmaceutical applications, drug discovery, telesurgery, and the like.

Another application for the sensor-based fluid detection device in engine fluids is an oil/antifreeze monitor, engine diagnostics for air/fuel optimization, diesel fuel quality, volatile organic carbon measurement (VOC), fugitive gases in refineries, food quality, halitosis, soil and water contaminants, air quality monitoring, fire safety, chemical weapons identification, use by hazardous material teams, explosive detection, breathalyzers, ethylene oxide or anesthetics detectors.

In another embodiment, there are provided systems for detecting an analyte in a sample. Such systems include, an array comprising more than one nanocluster; a sample containing at least one analyte; a Raman spectrometer; and a computer including an algorithm for analysis of the sample.

A variety of analytical techniques can be used to analyze the COIN particles described herein. Such techniques include for example, nuclear magnetic resonance spectroscopy (NMR), photon correlation spectroscopy (PCS), IR, surface plasma resonance (SPR), XPS, scanning probe microscopy (SPM), SEM, TEM, atomic absorption spectroscopy, elemental analysis, UV-vis, fluorescence spectroscopy, and the like.

In the practice of the present invention, the Raman spectrometer can be part of a detection unit designed to detect and quantify nanoclusters of the present invention by Raman spectroscopy. Methods for detection of Raman labeled analytes, for example nucleotides, using Raman spectroscopy are known in the art. (See, e.g., U.S. Pat. Nos. 5,306,403; 6,002,471; 6,174,677). Variations on surface enhanced Raman spectroscopy (SERS), surface enhanced resonance Raman spectroscopy (SERRS) and coherent anti-Stokes Raman spectroscopy (CARS) have been disclosed.

A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams may be used. The excitation beam passes through confocal optics and a microscope objective, and is focused onto the flow path and/or the flow-through cell. The Raman emission light from the labeled nanoclusters is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, that includes an avalanche photodiode interfaced with a computer for counting and digitization of the signal.

Another example of a Raman detection unit is disclosed in U.S. Pat. No. 5,306,403, including a Spex Model 1403 doublegrating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam may be spectrally purified with a bandpass filter (Corion) and may be focused on the flow path and/or flow-through cell using a 6× objective lens (Newport, Model L6X). The objective lens may be used to both excite the Raman-active organic compounds of the nanoclusters and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art may be used for detection of the nanoclusters of the present invention, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In certain aspects of the invention, a system for detecting the nanoclusters of the present invention includes an information processing system. An exemplary information processing system may incorporate a computer that includes a bus for communicating information and a processor for processing information. The information processing and control system may further comprise any peripheral devices known in the art, such as memory, display, keyboard and/or other devices.

In particular examples, the detection unit can be operably coupled to the information processing system. Data from the detection unit may be processed by the processor and data stored in memory. Data on emission profiles for various Raman labels may also be stored in memory. The processor may compare the emission spectra from composite organic-inbrganic nanoclusters in the flow path and/or flow-through cell to identify the Raman-active organic compound. The processor may analyze the data from the detection unit to determine, for example, the sequence of a polynucleotide bound by a probe of the nanoclusters of the present invention. The information processing system may also perform standard procedures such as subtraction of background signals

While certain methods of the present invention may be performed under the control of a programmed processor, in alternative embodiments of the invention, the methods may be fully or partially implemented by any programmable or hardcoded logic, such as Field Programmable Gate Arrays (FPGAs), TTL logic, or Application Specific Integrated Circuits (ASICs). Additionally, the disclosed methods may be performed by any combination of programmed general purpose computer components and/or custom hardware components.

Following the data gathering operation, the data will typically be reported to a data analysis operation. To facilitate the analysis operation, the data obtained by the detection unit will typically be analyzed using a digital computer. Typically, the computer will be appropriately programmed for receipt and storage of the data from the detection unit as well as for analysis and reporting of the data gathered.

In certain embodiments of the invention, custom designed software packages may be used to analyze the data obtained from the detection unit. In alternative embodiments of the invention, data analysis may be performed, using an information processing system and publicly available software packages.

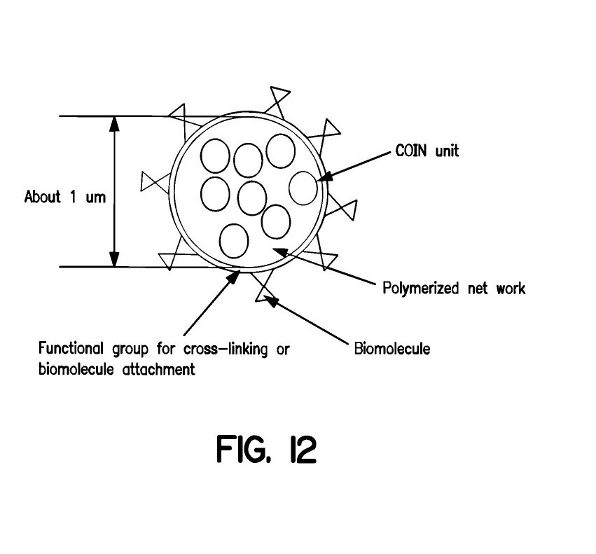

In other embodiments of the invention, there are provided microspheres comprising a plurality of COINs embedded and held together within a polymeric bead. Such microspheres can produce stronger and more consistent SERS signals than individual COINs. The polymer coating of the large microsphere can also provide surface areas for attachment of biomolecules, such as probes. The structural features are a) a structural framework formed by polymerized organic compounds; b) multiple COINs embedded in each micro-sized particle; c) a surface with suitable functional groups for attachment of desired functional groups, such as linkers, probes, and the like (FIG. 12). Several methods for producing microspheres according to this embodiment are set forth below.

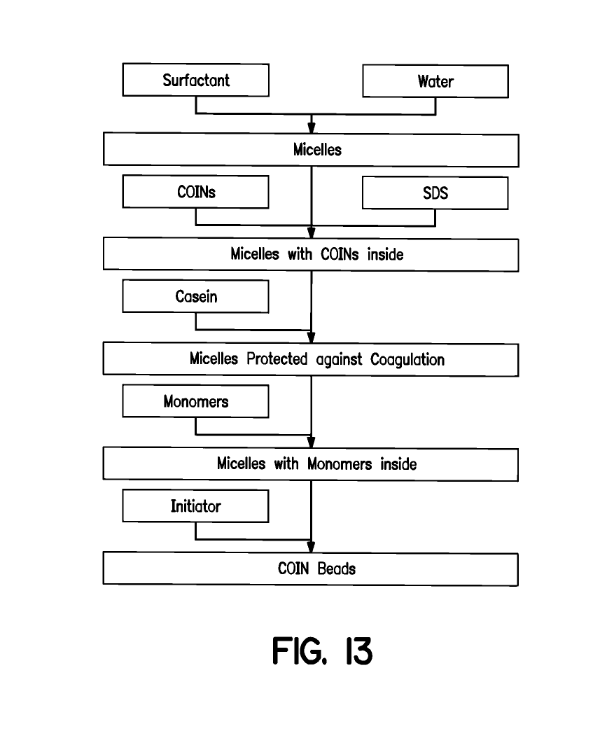

Inclusion method (FIG. 13): This approach employs the well established emulsion polymerization technique for preparing uniform latex microspheres except that COINs are introduced into the micelles before polymerization is initiated. As shown in the flow chart of FIG. 13, this aspect of the invention methods involves the following steps: 1) Micelles of desired dimensions are first prepared by homogenization of water with surfactants (e.g., octanol). 2) COIN particles are introduced along with a hydrophobic agent (e.g., SDS). The latter facilitates the transport of COINs into the interior of micelles. 3) Micelles are protected against aggregation with a stabilizing agent (e.g., Casein). 4) Monomers (e.g., styrene or methyl methacrylate) are introduced. 5) Finally, a free radical initiator (e.g., peroxide or persulfate) is used to start the polymerization to produce COIN embedded latex beads.

Additionally, COINs which have been embedded within a solid organic polymer bead can be used to form a microsphere. The polymer of the bead can prevent direct contact between COINs in the micelles and in the final product (microsphere). Furthermore, the number of COINs in each bead can be adjusted by varying the polymer thickness in the interstices of the bead. The polymer material of the bead is not needed for signal generation, the function of the polymer being structural.

The microspheres are up to microns in size and each operates as a functional unit having a structure comprising many individual COIN particles held together by the structural polymer of the bead. Thus, within a single microsphere are several COINs embedded in the structural polymer, which is the main inner and outer structural material of the bead. The structural polymer also functions as a surface attaching linkers, derivatives or for functional ization for attachment of probes. Since each COIN comprises a cluster of primary metal particles with at least one Raman-active organic compound adsorbed on the metal particles, the polymer of the bead for the most part does not come into contact with and hence does not attenuate Raman-activity of the Raman-active organic compounds which are trapped as they were adsorbed during colloid formation in the junctions of the primary metal particles of the COIN structure. Those Raman-active organic molecules on the periphery of the COIN that may come into contact with the structural polymer of the microsphere have reduced effect as Raman-active molecules.

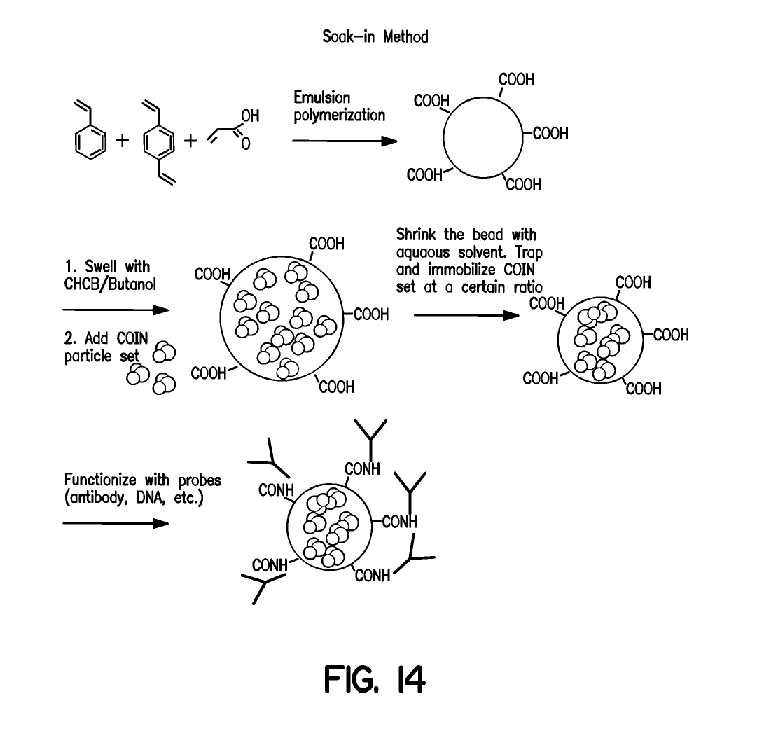

Soak-in method (FIG. 14): Microspheres are obtained first and allowed to contact COINs that are synthesized separately. Under certain conditions, such as in an organic solvent, the pores of the beads are enlarged enough to allow COINs to diffuse inside. After the liquid phase is changed to an aqueous phase, the pores of the bead close, embedding the COINs within the polymer beads. For example, 1) Styrene monomers are co-polymerized with divinylstyrene and acrylic acid to form uniformly-sized beads through emulsion polymerization. 2) The beads are swelled with organic solvents such as chloroform/butanol, and a set of COINs at a certain ratio are introduced so that the COINs diffuse into the swollen bead. 3) The beads are then placed in a non-solvent to shrinks the beads so that the COINs are trapped inside to form stable, uniform COIN-encapsulated beads.

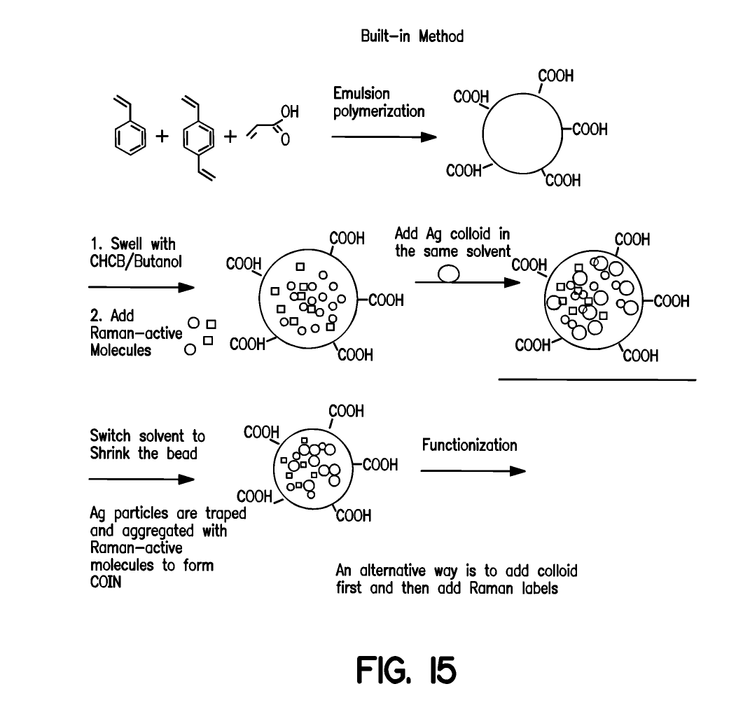

Build-in method (FIG. 15): In this method, microsphere beads are obtained first and are placed in contact with Raman labels and silver particles in organic solvents. Under this condition, the pores of the beads are enlarged enough to allow the labels and silver particles to diffuse inside. Then COIN clusters are formed inside the microsphere beads when silver colloids encounter each other in the presence of organic Raman labels. Heat and light can be used to accelerate aggregation and fusion of silver particles. Finally, the liquid phase is changed to aqueous phase, and the COINs are encapsulated. For example, 1) Styrene monomers are co-polymerized with divinylstyrene and acrylic acid to form uniformly-sized beads through emulsion polymerization. 2) The beads are then swelled with organic solvents such as chloroform/butanol, and a set of Raman-active molecules (for example, 8-aza-adenine and N-benzoyladenine) at a certain ratio is introduced so that the molecules diffuse into the swollen bead. Ag colloid suspension in the same solvent is then mixed with the beads to form Ag particle-encapsulated beads. 3) The solvent was switched to one that shrinks the beads so that the Raman labels and Ag particles are trapped inside. The process can be controlled so that the Ag particles will contact each other with Raman molecules in the junction, forming COINs inside the beads. When medium size silver colloids, such as, for example, 60 nm colloids, are used Raman labels are added separately (before or after silver addition) to induce colloid aggregation (formation of COINs) inside the beads. When 1-10 nm colloids are used, the labels can be added together. Then light or heat is used to induce the formation of active COINs inside the beads.

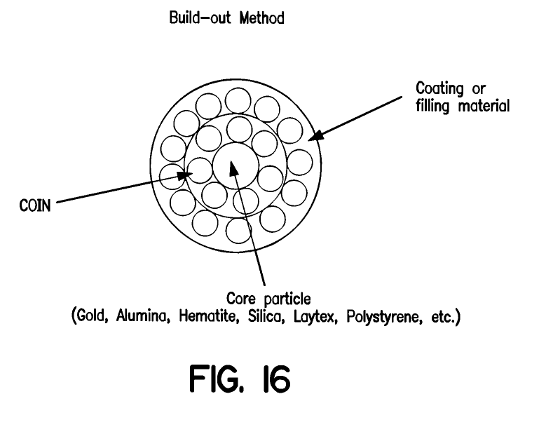

Build-out method (FIG. 16): In this method, a solid core is used first as the support for COIN attachment. The core can be metal (gold and silver), inorganic (alumina, hematite and silica) or organic (polystyrene, latex) particles. Attachment of COINs to the core particle can be induced by electrostatic attraction, van der Waals forces, and/or covalent binding. After the attachment, the assembly can be coated with a polymer to stabilize the structure and at the same time to provide a surface with functional groups. Multiple layers of COINs can be built based on the above procedure. The dimension of COIN beads can be controlled by the size of the core and the number of COIN layers. For example, 1) positively charged Latex particles of 0.5 μm are mixed with negatively charged COINs, 2) the Latex-COIN complex is coated with a cross-linkable polymer such as poly-acrylic acid. 3) The polymer coating is cross-linked with linker molecules such as lysine to form an insoluble shell. Remaining (unreacted) carboxylic groups would serve as the functional groups for second layer COIN attachment or probe attachment. Additional functional groups can also be introduced through co-polymerization or during the cross-link process.

### Example 1

### General Considerations

Chemical reagents: Biological reagents including anti-IL-2 and anti-IL-8 antibodies were purchased from BD Biosciences Inc. The capture antibodies were monoclonal antibodies generated from mouse, and the detection antibodies were polyclonal antibodies generated from mouse and conjugated with biotin. Liquid salt solutions and buffers were purchased from Ambion, Inc. (Austin, Tex., USA), which includes 5 M NaCl, 10×PBS (1×PBS 137 mM NaCl, 2.7 mM KCl, 8 mM Na₂HPO₄, and 2 mM KH₂PO₄, pH 7.4). Unless otherwise indicated, all other chemicals were purchased, at highest available quality, from Sigma Aldrich Chemical Company (St. Louis, Mo., USA). Deionized water used for experiments had a resistance of 18.2×10⁶ Ohms-cm that was obtained with a water purification unit (Nanopure Infinity, Barnstead, USA).

Silver seed particle synthesis: Stock solutions (0.50 M) of silver nitrate (AgNO₃) and sodium citrate (Na₃Citrate) were filtered twice through 0.2 micron polyamide membrane filters (Schleicher and Schuell, N.H., USA) which were thoroughly rinsed before use. Sodium borohydrate solution (50 mM) was made freshly and used within 2 hours after preparation. Silver seed particles were prepared by rapid addition of 50 mL of Solution A (containing 8.00 mM sodium citrate, 0.60 mM sodium borohydrate and 2.00 mM sodium hydroxide) into 50 mL of Solution B (containing 4.00 mM silver nitrate) under vigorous stirring. Addition of Solution B into Solution A led to a more polydispersed suspension. Silver seed suspensions were stored in the dark and were used within one week after preparation. Before use, the suspension was analyzed by Photon Correlation Spectroscopy (PCS, Zetasizer 3000 HS, Malvern) to ensure the intensity—averaged diameter (z-average) was between 10-12 nm with a polydispersity index less than 0.25.

Gold seed synthesis: A household microwave oven (1350W, Panasonic) was used to prepare gold nanoparticles. Typically, 40 mL of an aqueous solution containing 0.5 mM HAuCl₄ and 2.0 mM sodium citrate in a glass bottle (100 mL) was heated to boiling in the microwave using the maximum power, followed by a lower power setting to keep the solution gently boiling for 5 min. 2.0 grams of PTFE boiling stones (6 mm, Saint-Gobain A1069103, through VWR) were added to the solution to promote gentle and efficient boiling. The resultant solutions had a rosy red color. Measurements by PCS showed that the gold solutions had a typical z-average of 13 nm with a polydispersity index of <0.04.

COIN synthesis: alternative methods can be used.

Reflux method: To prepare COIN particles with silver seeds, typically, a 50 mL silver seed suspension (equivalent to 2.0 mM Ag⁺) was heated to boiling in a reflux system before introducing Raman labels. Silver nitrate stock solution (0.50 M) was then added drop-wise or in small aliquots (50-100 μL) to induce the growth and aggregation of silver seed particles. Up to a total of 2.5 mM silver nitrate could be added. The solution was kept boiling until the suspension became very turbid with a dark brown color. At this point, the temperature was lowered quickly by transferring the colloid solution into a glass bottle and then storing it at room temperature. The optimum heating time depended on the nature of Raman labels and amounts of silver nitrate addition. It was found helpful to verify that particles had reached a desired size range (for example, 80-100 nm on average) by PCS or UV-Vis spectrophotometer before the heating was arrested. Normally, the dark brown color was the indication of cluster formation and associated Raman activity.

To prepare COIN particles with gold seeds, typically, gold seeds were first prepared from 0.25 mM HAuCl₄ in the presence of a Raman label (e.g., 20 μM 8-aza-adenine). After heating the gold seed solution to boiling, silver nitrate and sodium citrate stock solutions (0.50 M) were added, separately, so that the final gold suspension contained 1.0 mM AgNO₃ and 1.0 mM sodium citrate. Silver chloride precipitate might form immediately after silver nitrate addition but disappeared soon with heating. After boiling, an orange-brown color developed and stabilized and an additional aliquot (50-100 μL) of silver nitrate and sodium citrate stock solutions (0.50 M each) was added to induce the development of a green color, which was the indication of cluster formation and was associated with Raman activity.

The procedures produced COINs with different colors, primarily due to the differences in the size of primary particles before cluster formation.

Oven method: COINs could also be prepared conveniently by using a convection oven. Silver seed suspension was mixed with sodium citrate and silver nitrate solutions in a 20 mL glass vial. The final volume of the mixture was typically 10 mL, which contained silver particles (equivalent to 0.5 mM silver ions), 1.0 mM silver nitrate and 2.0 mM sodium citrate (including the portion from the seed suspension). The glass vials were incubated in the oven set at 95° C. for 60 min. before being stored at room temperature. A range of label concentrations could be tested at the same time. Batches showing brownish color with turbidity were tested for Raman activity and colloidal stability. Batches with significant sedimentation (occurred when the label concentrations were too high) were discarded. Occasionally, batches that did not show sufficient turbidity could be kept at room temperature for an extended period of time (up to 3 days) to allow cluster formation. In many cases, suspensions became more turbid over time due to aggregation, and strong Raman activity developed within 24 hours. A stabilizing agent, such as bovine serum albumin (BSA), could be used to stop the aggregation and stabilize the COIN particles.

A similar approach was used to prepare COINs with gold cores. Briefly, 3 mL of gold suspension (0.50 mM Au³⁺), prepared in the presence of Raman labels, was mixed with 7 mL of silver citrate solution (containing 5.0 mM silver nitrate and 5.0 mM sodium citrate before mixing) in a 20 mL glass vial. The vial was placed in a convection oven and heated to 95° C. for 1 hour. Different concentrations of labeled gold seeds could be used simultaneously in order to produce batches with sufficient Raman activities. It should be noted that a COIN sample can be heterogeneous in terms of size and Raman activity. We typically used centrifugation (200-2,000×g for 5-10 min) or filtration (300 kDa, 1000 kDa or 0.2 micron filters, Pall Life Sciences through VWR) to enrich for particles in the range of 50-100 nm. The COIN particles can be coated with, for example, BSA or an antibody before enrichment. Some lots of COINs that we prepared (with no further treatment after synthesis) were stable for more than 3 months at room temperature without noticeable changes in physical and chemical properties.

Cold Method: 100 mL of silver particles (1 mM silver atoms) were mixed with 1 mL of Raman label solution (typically 1 mM). Then, 5 to 10 mL of 0.5 M LiCI solution was added to induce silver aggregation. As soon as the suspension became visibly darker (due to aggregation), 0.5% BSA was added to inhibit the aggregation process. Afterwards, the suspension was centrifuged at 4500 g for 15 minutes. After removing the supernatant (mostly single particles), the pellet was resuspended in 1 mM sodium citrate solution. The washing procedure was repeated for a total of three times. After the last washing, the resuspended pellets were filtered through 0.2 μM membrane filter to remove large aggregates. The filtrate was collected as COIN suspension. The concentrations of COINs were adjusted to 1.0 or 1.5 mM with 1 mM sodium citrate by comparing the absorbance at 400 nm with 1 mM silver colloids for SERS.

It should be noted that a COIN sample can be heterogeneous in terms of size and Raman activity. We typically used centrifugation (200-2,000×g for 5-10 min.) or filtration (300 kDa, 1000 kDa, or 0.2 micron filters, Pall Life Sciences through VWR) to enrich for particles in the range of 50-100 nm. COIN particles can be coated with a protection agent (for example, BSA, antibody) before enrichment. Some lots of COINs that we prepared (with no further treatment after synthesis) were stable for more than 3 months at room temperature without noticeable changes in physical and chemical properties.

Particle size measurement: The sizes of silver and gold seed particles as well as COINs were determined by using Photon Correlation Spectroscopy (PCS, Zetasizer3 3000 HS or Nano-ZS, Malvern). All measurements were conducted at 25° C. using a He—Ne laser at 633 nm. Samples were diluted with DI water when necessary. TEM analysis: for transmission electronic microscopic (TEM) analysis, carbon coated copper grids were used for sample preparation. The sample suspensions were sprayed on to the grid using an all-glass nebulizer (Ted Pella). Alternatively, a drop (20 μL) of sample suspension was deposited on the grid. After five minutes, the drop was blotted off with a piece of filter paper. Then the grid was allowed to touch the surface of a DI water drop for a few seconds to remove salts before drying in the air. TEM observation was made by using either JEM 2010 or 2010F with a UHR pole (Japan Electron Optics Laboratories). SEM analysis: for scanning electron microscopic (SEM) analysis, COIN particles were examined under a scanning electron microscope (S-4500, Hitachi). The sample preparation procedure was as follows: a small piece of silicon wafer substrate (1×1 cm2) was wet with a drop (20 μL) of poly-L-lysine (0.1%); after 5 min, the substrate was rinsed with deionized water (DI-water) and dried under a stream of nitrogen; a 20 μL of colloidal sample was then deposited on the poly-L-lysine-coated substrate. The substrate was finally rinsed with DI-water and let dry in air before SEM observation. Raman spectral analysis: for all SERS and COIN assays in solution, a Raman microscope (Renishaw, UK) equipped with a 514 nm Argon ion laser (25 mW) was used. Typically, a drop (50-200 μL)of a sample was placed on an aluminum surface. The laser beam was focused on the top surface of the sample meniscus and photons were collected for 10-20 second. The Raman system normally generated about 600 counts from methanol at 1040 cm-1 for 10 second collection time. For Raman spectroscopy detection of analyte immobilized on surface, Raman spectra were recorded using a Raman microscope built in-house. This Raman microscope consisted of a water cooled Argon ion laser operating in continuous-wave mode, a dichroic reflector, a holographic notch filter, a Czerny-Turner spectrometer, and a liquid nitrogen cooled CCD (charge-coupled device) camera. The spectroscopy components were coupled with a microscope so that the microscope objective focused the laser beam onto a sample, and collected the back-scattered Raman emission. The laser power at the sample was ˜60 mW. All Raman spectra were collected with 514 nm excitation wavelength.

Absorption spectral analysis: Extinction spectra for Raman labels and colloidal suspensions were recorded by an UV-Vis spectrophotometer (Model 8453, Agilent Technologies).

Conjugation of COIN particles with antibodies: a 500 μLt solution containing 2 ng of a biotinylated anti-human IL-2 or IL-8 antibody (anti-IL-2 or anti-IL-8) in 1 mM sodium citrate (pH 9) was mixed with 500 μL of a COIN solution (made with 8-aza-adenine or N-benzoyl-adenine); the resulting solution was incubated at room temperature for 1 hour, followed by adding 100 μL of PEG-400 (polyethylene-glycol-400). The solution was incubated at room temperature for another 30 min, and then 200 μL of 1% Tween-20® was added to the solution. The solution was centrifuged at 2000×g for 10 min. After removing the supernatant, the pellet was resuspended in 1 mL solution containing 0.5% BSA, 0.1% Tween-20 and 1 mM sodium citrate (BSAT). The solution was then centrifuged at 1000×g for 10 min. The BSAT washing procedure was repeated for a total of 3 times. The final pellet was resuspended in 700 μL of diluting solution (0.5% BSA, 1×PBS, 0.05% Tween-20®). The Raman activity of COIN was measured and adjusted to a specific activity of about 500 photon counts per μL per 10 seconds using a Raman microscope that generated about 600 counts from methanol at 1040 cm⁻¹ for 10 second collection time.

Confirmation of antibody-COIN conjugation: To obtain a standard curve, ELISA (enzyme-linked immunosorbent assay) experiments were performed according to manufacturer's instruction (BD BioSciences), using immobilized capture antibody, fixed analyte concentration (5 ng/mL IL-2 protein), and a serially diluted detection antibody (0, 0.01, 0.1, 1, and 10 μg/mL). After detection antibody binding, streptavidin-HRP (Horse Radish Peroxidase) was then reacted with the biotinylated detection antibodies and TMB (Tetramethyl Benzidine) substrate was applied followed by UV absorption measurement. A standard curve was generated by plotting absorption values against antibody concentrations. To estimate the amount of antibody molecules that could be attached to a COIN particle, a similar ELISA experiment was then performed with COIN conjugated with a detection antibody. The ELISA data were collected and the binding activity of the COIN-antibody conjugate was compared with the standard curve to estimate the equivalent amount of antibody in the COIN-antibody conjugate. Assuming that only one of the antibody molecules that had been conjugated to a COIN particle bound to an immobilized analyte, and that all biotin moieties associated with the COIN particle were bound by streptavidin-HRP. Finally, the number of antibody molecules per COIN was estimated by dividing the equivalent amount of antibody in the COIN-antibody by the estimated number of COIN particles. We estimated that there could be as many as 50 antibody molecules on a COIN particle.

Immuno sandwich assays: (I) Assay support Preparation: Xenobind™ Aldehyde slides (Polysciences, Inc., Pa., USA) were used as substrates for immuno assays; before being used, wells on a slide were prepared by overlaying a piece of cured PDMS of 1 mm thick (D. Duffy, J. McDonald, O. Schueller, and G. Whitesides, Rapid Prototyping of Microfluidic Systems in Poly(dimethylsiloxane). Anal. Chem., 1998. 70(23): p. 4974-4984). The PDMS had holes of 5 mm in diameter. (2) Capture antibody binding: Anti-human IL-2 antibody (9 ug/mL) was prepared in 0.33×PBS. An aliquot of 50 μL of the antibody was added to wells on the slide and the slide was incubated in a humidity chamber at 37° C. for 2 hours. (3) Surface blocking: After removing antibody solution, 50 μL of 1% BSA in a 10 mM glycine solution was added to each well to quench the aldehyde groups. The slide was incubated at 37° C. for another 1 hour, then the wells were washed 4 times, each with 50 μL PBST washing solution (1×PBS, supplemented with 0.05% Tween-20®). (4) Protein binding: IL-2 and IL-8 protein solutions at various concentrations (from 0-50 μg/mL, depending on experiments) were prepared in dilution buffer (1×PBS, 0.5% BSA, 0.05% Tween-20). A sample containing 404 of an antibody solution was added to a well; binding was carried out at 37° C. for several hours (over night was preferred to ensure complete binding). The sample-containing wells were washed with 50 μL of PBST solution for a total of 4 times. (5) Detection antibody binding: equal amounts of COIN samples conjugated with anti-IL-2 detection antibody and anti-IL-8 detection antibody, respectively, were combined and then added to each PDMS well; the solutions were then incubated at 37° C. for 1 hour. After removing the conjugate solutions, the wells were washed four times, each with 50 μL of dilution buffer solution, followed by washing with 50 μL of DI water once. Finally, 30 μL of DI-water was added to each well before Raman signal detection.

### Example 2

COIN synthesis and analysis. Silver colloidal solution (50 mL) with average particle diameter of 12 nm was made from 2 mM AgNO₃, 0.3 mM NaBH₄ and supplemented with 4 mM Na₃Citrate. The solution was heated to boil before 8-aza-adenine (AA) was added to final 20 μM. After 5 min. of boiling, additional 0.5 mM AgNO₃ was added. The temperature was then lowered and maintained at 95+1° C. Aliquots (1 mL each) of the solution were retrieved at indicated time intervals for spectral measurements after 1:30 dilution with 1 mM sodium citrate. As shown in FIG. 2A, absorption spectra of retrieved sample aliquots, showed peak shifts and increased absorption at higher wavelengths (>450 nm). At time intervals (small arrows indicate positions where absorption changes were further analyzed) retrieved sample aliquots (each 50 placed in a Petri-dish over a white light box), were photographed and showed time dependent-color changes with reaction heating time. Absorbance and Raman activity as a function of reaction (heating) time are shown in FIG. 2B. A decrease in absorbance at 700 nm after 65 min. was caused by the formation of large aggregates that settled quickly in solution.

### Example 3

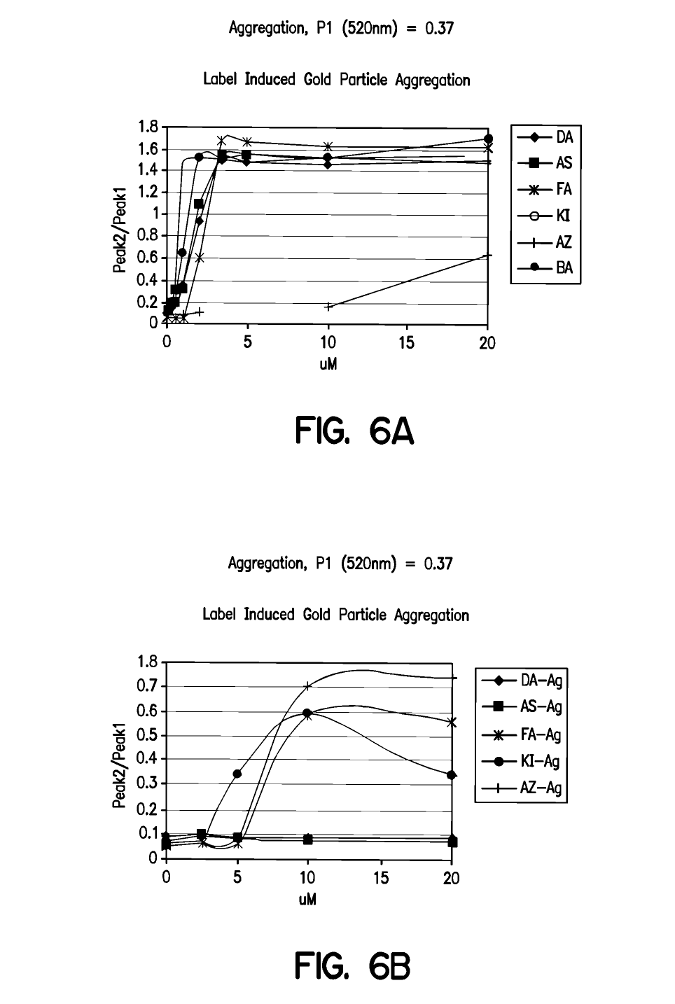

Organic compound-induced metal particle aggregation: Using metal particles prepared as described herein (gold of 15 nm, Abs_{520 nm}=0.37; silver of 60 nm, Abs_{420 nm}=0.3) in 1 mM Na₃Citrate; each organic compound (see key to abbreviations in Table 1) was mixed with a sample of a metal colloid solution at indicated concentrations for 10 min. before spectral measurement. For each sample, the absorbance of the main peak was used as the Peak 1 value and the increased absorbance at a higher wavelength (600 nm-700 nm) was used as the Peak 2 value; the ratios of Peak 2/Peak 1 were plotted against concentrations of the organic compound; a high value of the ratio indicating a high degree of metal particle aggregation. FIG. 6A shows aggregation of gold particles induced by organic compounds. Relatively low concentrations of organic compounds were sufficient to cause aggregation of silver particles. As shown in FIG. 6B, comparatively high concentrations of organic compounds were required to induce aggregation of silver particles.

### Example 4

Zeta potential of silver particles as a function of 8 aza-adenine concentration: Silver particles were prepared by reduction of silver nitrate with sodium citrate at 95° C.-100° C. The z-average size of the particles as determined by PCS (Zetasizer Nano-ZS, Malvern) was 47 nm. The total silver concentration was fixed at 0.10 mM with a suspending medium of 1.00 mM sodium citrate for the zeta potential measurement. Using the same silver concentration and suspending medium, the evolution of aggregate size (z-average) in the presence of 20 μM 8-aza-adenine was measured. FIGS. 7A and B show, respectively, the absolute zeta potential and aggregation kinetics. A higher absolute zeta potential and slower aggregation kinetics were expected under COIN synthesis conditions where much higher silver concentrations (1-4.5 mM) and smaller particles (less than 20 nm) were used.

### Example 5

TEM analysis of silver particles was conducted under four conditions of preparation: Silver colloids were synthesized by methods described herein. 1. The sample was kept at room temperature for I week before being analyzed by transmission electronic microscopy (TEM), which showed that most particles were less than 10 nm. 2. A silver sample from the same source was boiled for 40 min. and then cooled to room temperature before TEM analysis, which showed no obvious change in the particle size. 3. A silver sample from the same source was incubated with 8-aza-adenine (final concentration of 20 μM) for two weeks at room temperature before TEM analysis, which showed that some particles had started to aggregate and fuse. 4. Silver particles analyzed by TEM after boiling for 19 min. in the presence of 20 μM 8-aza-adenine showed the appearance of small particles (less than 10 nm) and of large particles (greater than 10 nm). These results (see also FIG. 2) lead to the conclusion that extended boiling would cause cluster formation.

### Example 6

Synthesis of COINs coated with BSA

Coating Particles with BSA: COIN particles were coated with, an adsorption layer of BSA by adding 0.2% BSA to the COIN synthesis solution when the desired COIN size was reached. The addition of BSA inhibited further aggregation.

Crosslinking the BSA Coating: The BSA adsorption layer was crosslinked with glutaraldehyde followed by reduction with NaBH₄. Crosslinking was accomplished by transferring 12 mL of BSA coated COINs (having a total silver concentration of about 1.5 mM) into a 15 mL centrifuge tube and adding 0.36 g of 70% glutaraldehyde and 213 μL of 1 mM sodium citrate. The solution was mixed well and allowed to sit at room temperature for about 10 min. before it was placed in a refrigerator at 4° C. The solution remained at 4° C. for at least 4 hours and then 275 μL of freshly prepared NaBH₄ (1 M) was added. The solution was mixed and left at room temperature for 30 min. The solution was then centrifuged at 5000 rpm for 60 min. The supernatant was removed with a pipet leaving about 1.2 mL of liquid and the pellet in the centrifuge tube. The COINs were resuspended by adding 0.8 mL of 1 mM sodium citrate to yield a final volume of 2.0 mL.

FPLC Purification of Encapsulated COINS: The coated COINs were purified by FPLC (fast protein liquid chromatography) on a crosslinked agarose size-exclusion column. The concentrated COIN reaction mixture suspension (2.0 mL) was purified with a Superose 6 FPLC column on an AKTA Purifier. The COIN mixture was injected in 0.5 ml batches and an isocratic flow of 1 mM sodium citrate at 1 ml/min. was applied to the column. Absorbance at 215 nm, 280 nm, and 500 nm was monitored for peak collection. The encapsulated COINs eluted at about 7-9 min., while the BSA/crosslinked BSA fraction eluted at about 9-11 min. Glutaraldehyde, sodium borohydride, and Raman labels eluted after about 20 min. Fractions from multiple FPLC runs were combined.

### Example 6

Electron micrographs show effect of cluster formation on Raman signals of COINs.

1. Transmission electronic microscopy (TEM) analysis of silver seeds as the starting material, showed most particles were <10 nm; no SERS effect was detected.

2. TEM of enlarged silver particles formed by heating silver seed particles in the presence of organic Raman labels (in this particular sample, the Raman labels were 2.5 μM 8-aza-adenine, 5.0 μM methylene blue and 2.5 μM 9-amioacridine, showed most particles were >10 nm with very few clusters; other Raman labels gave similar results); Raman signals were weak.

3. TEM of Raman-active clustered nanoparticles, made under similar conditions as in 2, except that higher Raman label concentrations (5.0 μM 8-aza-adenine, 5.0 μM methylene blue and 7.5 μM 9-aminoacridiene) showed formation of a large amount of clusters and strong Raman signal was detected from this sample even though the sample would give weak Raman signal before clusters were formed.

4. Gold seed particles with similar size and morphology were made in the presence of Raman labels (e.g., 10 μM adenine or 20 μM 8-aza-adenine).

5. Silver particles with gold cores (made from a solution containing 0.25 mM AuHCl₄ and 1.25 mM AgNO₃); the gold cores were made from gold ions in the presence of 10 μM Adenine gave detectable Raman signals only when salt (i.e., 100 mM LiCI) was used to induce aggregation.

6. Scanning electronic micrograph showed Raman active silver clusters prepared with 5 μM N-benzoyl adenine under similar conditions as in 5, except that additional AgNO₃ (0.75 mM) was added to cause cluster formation.

### Example 7

Comparison of Raman signals of SERS and COIN. For SERS testing, 100 μL silver colloids containing 8-aza-adenine (AA, final 4 μM) was mixed with 100 μL of a test reagent chosen from the following: water (control), N-benzoyl adenine (BA, 10 μM), BSA (1%), Tween-20® (Twn, 1%), ethanol (eth, 100%); a resulting 200 μL mixture was then mixed with either 100 μL water (−Li,) or 100 μL of 0.34 M LiCI (+Li,) before Raman scattering signal was measured by a Raman microscope. Raman signals were in arbitrary unit and were normalized to respective maximums. The same procedure was used for testing COIN (made with 20 μM 8-aza-adenine), except that an additional 8-aza-adenine was not used. FIG. 9A shows SERS spectra of 8-aza-adenine (AA) with N-benzoyladenine (BA) as the test reagent, showing salt was required for the SERS signal and AA signal was suppressed by BA signal; FIG. 9B shows. Raman spectra from COIN using BA as the test reagent, indicating that salt was not required for production of COIN signal and that salt reduced AA signal. Only a weak BA signal was detected when salt was added. FIG. 9C shows SERS spectra of 8-aza-adenine (AA) with bovine serum albumin (BSA) as the test reagent, showing SERS signals were inhibited by BSA; FIG. 9D shows Raman spectra from COIN using BSA as the test reagent, indicating that BSA had little negative effect on COIN and might actually stabilized COIN. FIG. 9E shows SERS spectra of 8-aza-adenine (AA) with Tween-20® (Twn) as the test reagent, showing relatively strong SERS signal was detected in the absence of salt; FIG. 9F shows Raman spectra from COIN using Tween-20® as the test reagent, indicating that Tween-20 inhibited part of the COIN signal but, on the other hand, could compensate partially for the negative effect of salt; FIG. 9G shows SERS spectra of 8-aza-adenine (AA) with ethanol (Eth) as the test reagent, showing salt was required for the SERS signal and that 3 peaks (indicated by arrows) were enhanced by ethanol; FIG. 9H shows Raman spectra from COIN using ethanol as the test reagent, indicating that salt had a negative effect on COIN signal and that no enhanced peaks were noticeable.

### Example 8

Use of COINs as tags for multiplex analyte detection. Using a detection scheme as shown in FIG. 5A in which an amplification reaction step after analyte binding by antibody-conjugated COINs was eliminated, a set of 50 spectra were collected from an immuno sandwich assay for IL-2 using 8-aza-adenine COIN as the tag (FIG. 5B main peak position at 1340 cm₋₁). 40 μL of IL-2 at 1 pg/mL was added to a 5-mm well coated with immobilized IL-2 capture antibody; the 50 spectra were collected from one sample by continuously moving the motorized stage; each spectrum represents the information collected over a 100 millisecond period. The laser beam size was about 4 microns in diameter. Background signals were subtracted; spectra were offset in both X and Y axes to show individual spectra. FIG. 5C is a bar graph of analyte signals; experiments were carried out with samples containing 1 or 2 of the analytes IL2 and IL8 (both having molecular weights of about 20 kDa) at different ratios (5:0, 4:1, 1:1, 1:4 and 0:5); the samples were tested in separate vessels and the combined analyte concentration for each sample was 50 pg/mL; IL-2 detection antibody was conjugated to COIN prepared with 8-aza-adenine (AA) and IL-8 detection antibody was conjugated to COIN made with N-benzoyl adenine (BA) at a 1:1 ratio; data were collected from a total of 400 data points for each sample. Spectra showing positive signals at the expected Raman shift positions were counted as measured signal points (FIG. 5C; wide bars), and expressed as percentages of the total positive signals for both analytes in corresponding samples. Expected values (a total of 100% for the 2 labels) are shown as narrow bars (FIG. 5C).

Although the invention has been described with reference to the above example, it will be understood that modifications and variations are encompassed within the spirit and scope of the invention. Accordingly, the invention is limited only by the following claims.

What is claimed is:

1) A method for the synthesis of a nanocluster of metal particles having a unique Raman signature, wherein the unique Raman signature is produced by at least one Raman active organic compound that is incorporated within the nanocluster, the method comprising:
providing a mixture comprising at least one Raman-active compound and nanoparticles of a metal suspended in a liquid wherein the Raman-active compound is dissolved in the liquid;
generating nanoclusters of nanoparticles that incorporate the Raman-active compound and that have an average diameter of 20 nm to 200 nm from the liquid mixture, wherein generating clusters of nanoparticles comprises heating the liquid mixture, adding LiCl to the mixture, or a combination thereof.

2) The method for the synthesis of a nanocluster of claim 1 wherein the metal particles are comprised of a metal selected from group consisting of silver, gold, copper, palladium, platinum, and aluminum.

3) The method for the synthesis of a nanocluster of claim 1 wherein the metal particles are comprised of silver or gold.

4) The method for the synthesis of a nanocluster of claim 1 wherein the nanocluster has an average diameter of about 40 to about 200 nm.

5) The method for the synthesis of a nanocluster of claim 1 wherein the nanocluster has an average diameter of about 50 nm to 150 nm.

6) The method for the synthesis of a nanocluster of claim 1 wherein the mixture contains two different organic compounds capable of being detected by Raman spectroscopy and the resulting nanocluster has the two different organic compounds incorporated therein.

7) The method for the synthesis of a nanocluster of claim 1 also including attaching a probe to the nanocluster wherein the probe is selected from the group consisting of antibodies, antigens, polynucleotides, oligonucleotides, receptors, carbohydrates, cofactors, and ligands.

8) The method for the synthesis of a nanocluster of claim 1 also including coating the nanocluster with an adsorption layer wherein the adsorption layer comprises a protein.

9) The method for the synthesis of a nanocluster of claim 8 wherein the protein comprises bovine serum albumin.

10) The method for the synthesis of a nanocluster of claim 8 also including attaching a probe to the nanocluster wherein the probe is selected from the group consisting of antibodies, antigens, polynucleotides, oligonucleotides, receptors, carbohydrates, cofactors, and ligands.
